(19) United States
(12) Patent Application Publication
(10) Pub. No.: US 2006/0161994 A1
(43) Pub. Date: Jul. 20, 2006

(54) Functional assays for cholesterol absorption inhibitors

(75) Inventors: Diane Levitan, Tenafly, NJ (US); Marsha Smith, Princeton, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/301,094

(22) Filed: Dec. 12, 2005

Related U.S. Application Data
(60) Provisional application No. 60/636,390, filed on Dec. 15, 2004.

(51) Int. Cl. A01K 67/033

(52) U.S. Cl. 800/3; 800/13

(57) ABSTRACT

The present invention relates to functional assays performed in C.elegans worms that are useful for the identification of NPC1L1 inhibitors. Compositions useful for the performance of such assays are also provided.

This application claims the benefit of U.S. provisional patent application No. 60/636,390; filed Dec. 15, 2004 which is herein incorporated by reference in its entirety.

### FIELD OF THE INVENTION

The present invention relates to Caenorhabditis elegans cells along with methods of use thereof.

### BACKGROUND OF THE INVENTION

A factor leading to development of vascular disease, a leading cause of death in industrialized nations, is elevated serum cholesterol. It is estimated that 19% of Americans between the ages of 20 and 74 years of age have high serum cholesterol. The most prevalent form of vascular disease is arteriosclerosis, a condition associated with the thickening and hardening of the arterial wall. Arteriosclerosis of the large vessels is referred to as atherosclerosis. Atherosclerosis is the predominant underlying factor in vascular disorders such as coronary artery disease, aortic aneurysm, arterial disease of the lower extremities and cerebrovascular disease.

Cholesteryl esters are a major component of atherosclerotic lesions and the major storage form of cholesterol in arterial wall cells. Formation of cholesteryl esters is also a step in the intestinal absorption of dietary cholesterol. Thus, inhibition of cholesteryl ester formation and reduction of serum cholesterol can inhibit the progression of atherosclerotic lesion formation, decrease the accumulation of cholesteryl esters in the arterial wall, and block the intestinal absorption of dietary cholesterol.

The regulation of whole-body cholesterol homeostasis in mammals and animals involves the regulation of intestinal cholesterol absorption, cellular cholesterol trafficking, dietary cholesterol and modulation of cholesterol biosynthesis, bile acid biosynthesis, steroid biosynthesis and the catabolism of the cholesterol-containing plasma lipoproteins. Regulation of intestinal cholesterol absorption has proven to be an effective means by which to regulate serum cholesterol levels.

One protein that mediates cholesterol absorption in the intestine is the NPC1L1 protein. The NPC1L1 protein bears sequence similarity to several proteins including NPC1. Homologues of NPC1L1 have been identified in the nematode Caenorhabditis elegans.

The nematode C. elegans is a cholesterol auxotroph—under laboratory conditions, exogenous cholesterol must be added to the growth media for worms to survive (Chitwood, D. J. Crit. Rev. Biochem. Mol. Biol. 34: 273-284 (1999)). Thus, this organism can serve as a useful model for the transport, absorption and function of cholesterol (for review see Kurzchalia, T. V. and S. Ward Nature Cell Biology 5(8): 684-688 (2003)). The two potential homologs of the human NPC genes, in C.elegans, are called ncr-1 and ncr-1 (for Neimann-Pick C related; previously called npc-1 and npc-2). The NCR-1 and NCR-2 proteins are 27% identical to human NPC1 and 26% identical to human NPC1L1.

A role of ncr-1 and ncr-2 in C. elegans was determined by generating deletion mutations in both genes (Sym, M., M. Basson, et al. Current Biology 10(9): 527-530 (2000)). While both ncr-1 and ncr-2 mutants were displayed no obvious phenotype under standard growth conditions, ncr-1 mutants displayed a hypersensitivity to cholesterol deprivation, suggesting a role in cholesterol uptake or utilization. Furthermore, the ncr-1; ncr-2 double mutant displayed a novel phenotype-constitutive entry into the alternative life stage of dauer (the Daf-c phenotype), even under favorable conditions. Dauer larvae normally form only under conditions of crowding, starvation, or high temperature (Riddle, D. L. and P. S. Albert (1997). Genetic and environmental regulation of dauer larva development. C. elelgans II. D. L. Riddle, T. Blumenthal, B. J. Meyer and J. R. Priess. Cold Spring Harbor, Cold Spring Harbor Laboratory Press: 739-768.). Thus, while ncr-2 plays a more limited role in cholesterol sensitivity, ncr-1 and ncr-2 appeared to be functionally redundant with respect to dauer formation.

Epistasis analysis has placed ncr-1 and ncr-2 in an already defined genetic pathway which governs the animal's decision to become a dauer. ncr-1 and ncr-2 were shown to act upstream of daf-12, which encodes a nuclear hormone receptor whose signaling ability is necessary for dauer formation (Antebi, A., W. H. Yeh, et al. Genes Dev. 14(12): 1512-1527 (2000)) and daf-9, a cytochrome P450 like gene which is thought to be involved in the biosynthesis of the ligand for daf-12 (Gerisch, B., C. Weitzel, et al. Dev Cell 1(6): 841-851(2001); Jia, K., P. S. Albert, et al. Development 129(1): 221-231 (2002)). Since cholesterol deprivation can enhance weak daf-9 or daf-12 mutations ((Gerisch, B., C. Weitzel, et al. Dev Cell 1(6): 841-851(2001)), it is believed that a cholesterol moiety is important for the production of the daf-12 ligand. Thus, ncr-1 and ncr-2 play a role in cholesterol trafficking which feeds into the daf-9/daf-12 pathway.

A cholesterol absorption inhibitor, ezetimibe ( 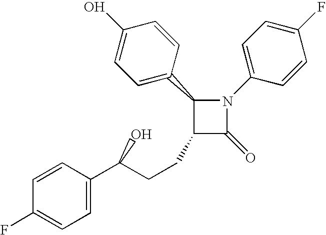
see U.S. Pat. Nos. 5,767,115 and RE37721), has been shown to be effective in the reduction of intestinal cholesterol absorption. A pharmaceutical composition containing ezetimibe is commercially available from Merck/Schering-Plough Pharmaceuticals, Inc. under the tradename Zetia®.

A cellular target through which ezetimibe acts, in humans, is the human NPC1L1 protein (U.S. Patent Application Publication No. 20040161838; PCT Published Patent Application No. WO2004/009772; Genbank Accession No. AF192522; Davies et al., (2000) Genomics 65(2):137-45 and Ioannou, (2000) Mol. Genet. Metab. 71(1-2):175-81)). Assays through which other agents that inhibit NPC1L1-mediated cholesterol absorption have been described (see e.g., U.S. Patent Application Publication No. 20040161838; PCT Published Patent Application No. WO2004/009772). However, there remains a need in the art for alternative, convenient, high-throughput functional assays through which NPC1L1 inhibitors can be identified.

### SUMMARY OF THE INVENTION

The present invention addresses, inter alia, the need in the art for convenient functional assays for identifying NPC1L1 inhibitors.

The present invention provides a method for identifying a substance that inhibits intestinal cholesterol absorption, reduces elevated total cholesterol, reduces elevated low density lipoprotein cholesterol, reduces elevated apolipoprotein B, treats or prevents heterozygous familial hypercholesterolemia, treats or prevents non-familial hypercholesterolemia, treats or prevents homozygous familial hypercholesterolemia, or treats or prevents homozygous sitosterolemia comprising: (a) contacting a C.elegans worm having a functional NPC1L1 polypeptide but lacking functional ncr-1 and ncr-2 polypeptide with the substance to be tested; and (b) determining if the worm exhibits a dauer phenotype; whereby the substance is identified as being an inhibitor of intestinal cholesterol absorption, capable of reducing elevated total cholesterol, capable of reducing elevated low density lipoprotein cholesterol, capable of reducing elevated apolipoprotein B, useful for treating or preventing heterozygous familial hypercholesterolemia, useful for treating or preventing non-familial hypercholesterolemia, useful for treating or preventing homozygous familial hypercholesterolemia, or useful for treating or preventing homozygous sitosterolemia if the dauer phenotype is observed. In an embodiment of the invention, the dauer phenotype is identified by visual inspection. In an embodiment of the invention NPC1L1 is human NPC1L1 (e.g., SEQ ID NO: 6).

The present invention also provides a method for identifying a substance that inhibits intestinal cholesterol absorption, reduces elevated total cholesterol, reduces elevated low density lipoprotein cholesterol, reduces elevated apolipoprotein B, treats or prevents heterozygous familial hypercholesterolemia, treats or prevents non-familial hypercholesterolemia, treats or prevents homozygous familial hypercholesterolemia, or treats or prevents homozygous sitosterolemia comprising: (a) contacting a C.elegans worm having a functional NPC1L1 polypeptide but lacking functional ncr-1 and ncr-2 polypeptide with the substance to be tested; and (b) determining whether the worm secretes chitinase; whereby the substance is identified as being an inhibitor of intestinal cholesterol absorption, capable of reducing elevated total cholesterol, capable of reducing elevated low density lipoprotein cholesterol, capable of reducing elevated apolipoprotein B, useful for treating or preventing heterozygous familial hypercholesterolemia, useful for treating or preventing non-familial hypercholesterolemia, useful for treating or preventing homozygous familial hypercholesterolemia, or useful for treating or preventing homozygous sitosterolemia if chitinase is not secreted. In an embodiment of the invention, chitinase is detected by measuring cleavage of the substrate 4-methylumbelliferyl-β-D-N,N′,N″-triacetylchito-trioside. In an embodiment of the invention, the NPC1L1 is human NPC1L1 (e.g., SEQ ID NO: 6).

The present invention also provides a method for identifying a substance that inhibits intestinal cholesterol absorption, reduces elevated total cholesterol, reduces elevated low density lipoprotein cholesterol, reduces elevated apolipoprotein B, treats or prevents heterozygous familial hypercholesterolemia, treats or prevents non-familial hypercholesterolemia, treats or prevents homozygous familial hypercholesterolemia, or treats or prevents homozygous sitosterolemia comprising: (a) contacting a C.elegans cell having a functional NPC1L1 polypeptide but lacking functional ncr-1 and ncr-2 polypeptide and having an adult-specific C.elegans promoter operably linked to a reporter with the substance to be tested; and (b) determining whether the expression by the promoter occurred; whereby the substance is identified as being an inhibitor of intestinal cholesterol absorption, capable of reducing elevated total cholesterol, capable of reducing elevated low density lipoprotein cholesterol, capable of reducing elevated apolipoprotein B, useful for treating or preventing heterozygous familial hypercholesterolemia, useful for treating or preventing non-familial hypercholesterolemia, useful for treating or preventing homozygous familial hypercholesterolemia, or useful for treating or preventing homozygous sitosterolemia if the expression is not detected. In an embodiment of the invention, the NPC1L1 is human NPC1L1 (e.g., SEQ ID NO: 6). In an embodiment of the invention, the adult-specific C.elegans promoter is a member selected from the group consisting of the col-19 promoter and the vit-2 promoter. In an embodiment of the invention, the reporter is a member selected from the group consisting of Photorhabdus luminescens LuxA-E, FMN oxidoredtuctase; amFP486; zFP506; zFP538; dsFP483; drFP583; cFP484; Pyrophorus plagiophthalamus luciferase; Chloramphenicol Acetyltransferase (CAT); β-Galactosidase (β-Gal); Vibrio harveyi luciferase; Photinus pyralis Luciferase; Renilla reniformis luciferase; Green Fluorescent Protein; β-glucuronidase (GUS) and chitinase.

The present invention also includes a method for producing NPC1L1 (e.g., human NPC1L1 such as SEQ ID NO: 6) comprising introducing a polynucleotide encoding NPC1L1 operably linked to a promoter into a C.elegans cell and propagating said cell and, optionally, isolating the NPC1L1 from the propagated cell.

The preset invention also provides a transgenic Caenorhabditis elegans worm whose cells lack functional ncr-1 protein and ncr-2 protein and have functional NPC1L1 protein. In an embodiment of the invention, the worm is strain N2 lacking functional ncr-1 and ncr-2 protein and having functional NPC1L1 polypeptide. In an embodiment of the invention, the NPC1L1 is human NPC1L1 (e.g., SEQ ID NO: 6). In an embodiment of the invention, the NPC1L1 polynucleotide is integrated into a C.elegans chromosome (e.g., I, II, Ill, IV, V or X). In an embodiment of the invention, the polynucleotide encoding NPC1L1 is operably associated with a promoter (e.g., the ncr-1 or ncr-2 promoter).

The scope of the present invention also includes an isolated transgenic C.elegans worm having functional NPC1L1 polypeptide (e.g., human NPC1L1 such as SEQ ID NO: 6). The NPC1L1 gene can be operably associated with a C.elegans promoter (e.g., the ncr-1 or ncr-2 promoter).

The present invention provides an isolated transgenic C.elegans worm that is selected from the group consisting of Strain 2a, Strain 2b, Strain 3, Strain 4a and Strain 4b (see infra).

The present invention further provides an isolated plasmid selected from the group consisting of ncr-1p/hNPC1L1/49.26; ncr-2p/hNPC1L1/49.26 and ncr-1p/GFP/49.26.

Also provided by the present invention is an isolated oligonucleotide selected from the group consisting of SEQ ID NOs: 7-14.

### DETAILED DESCRIPTION OF THE INVENTION

Human NPC1L1 can substitute for and complement ncr-1 and/or ncr-2. Described herein is a functional assay useful for screening for compounds that inhibit the function of NPC1L1 (e.g., human NPC1L1). The assays described herein are also useful for examining many questions addressing the structure/function relationship of NPC1L1 (e.g., human NPC1L1). NPC1L1 inhibitors identified using the screening assays of the present invention are useful, inter alia, for inhibiting intestinal cholesterol absorption, reducing total cholesterol, LDL cholesterol or apolipoprotein B and for treatment and prevention of cardiovascular disease including hyperlipidemia, hypercholesterolemia, sitosterolemia, atherosclerosis, coronary heart disease, stroke, arteriosclerosis and other diseases mediated or exacerbated by dietary cholesterol absorption.

### Molecular Biology

The meaning of ncr-1 and ncr-2 are well known in the art. In an embodiment, the ncr-1 and ncr-2 gene and protein sequences are as follows:

ncr-1 Spliced Coding Region (Genbank Accession No. F02E8.6):

| | |
|---|---|
| atgaaacaactactcattttttgcttgctatttgggt | (SEQ ID NO:1) |
| ctatattccatcatggcgacgcgggatgtatcatgcg | |
| aggattgtgccagaagcatactgaaaatgcatatgga | |
| ccatgtgttaccaacgatactaatgtggagcccacag | |
| cttttgacaaaactcatccggcatatgagaaaatggt | |
| cgagttttgcccccatttgctaactggtgacaacaaa | |
| ctctgctgtacgccatcgcaagcggaaggactgacta | |
| agcaaattgcacaagcccgacatattctgggacgatg | |
| tccgtcgtgcttcgacaactttgccaaattgtggtgc | |
| gaatttacgtgcagcccgaatcaacaggattttgtat | |
| cgatctccgaaatgaaaccaatcgagaagaaagaggg | |
| attcaccccagagtaccaaccagctgaggcatacgtg | |
| aatacagtcgagtacagattgtctactgattttgctg | |
| agggaatgttctcgtcttgtaaagatgtaacttttgg | |
| agggcagcctgcattacgagtcatgtgtacttctact | |
| ccgtgtactttgacaaattggttggaatttattggaa | |
| ctcaaaatctagatctcaatattccaatccacacaaa | |
| gttccttctctatgaccctatcaagacacctccatcg | |
| gatcgttcaacgtacatgaatgtcaactttactgggt | |
| gcgacaaatcagctcgagtcggctggccggcttgttc | |
| aacgtcagagtgcaacaaggaagagtacgcaaacttg | |
| atcgacctggacgatggaaaaacttctgggcaaactt | |
| gcaatgttcatggcatcgcctgcctgaacatttttgt | |
| catgctcgccttcatcggctcacttgccgtgcttctt | |
| tgtgttggatttgtattcacatcatatgacgaagact | |
| acacgaacttgaggcaaacacaatctggagaagaatc | |
| cccgaaacggaacagaatcaaacggaccggtgcctgg | |
| attcacaacttcatggagaacaatgctcgagatattg | |
| gaatgatggcgggacgcaatccaaagtctcacttttt | |
| cataggatgtgccgttctgattttctgcttgccagga | |
| atgatttatcacaaagagtcgacaaatgttgttgata | |
| tgtggtcttctccgagatccagggctcgacaagaaga | |
| gatggttttcaatgccaactttggacgcccacagaga | |
| tatcagcaaattatgttgctaagtcaccgcgatttcc | |
| aatcgagcgggaagctttatggaccagtattccacaa | |
| ggacatttttgaggagcttttcgatattttaaatgcc | |
| atcaaaaatatatcaacgcaagactctgatggtcgga | |
| caatcacacttgatgatgtatgttatcgacctatggg | |
| accaggatatgattgtttgattatgtctccaacgaac | |
| tattttcaagggaacaaagagcatctggatatgaaat | |
| cgaacaaagaagaaacggtttccgaagatgatgacgc | |
| gtttgattatttctcatcggaagcaacaacagatgaa | |
| tggatgaatcacatggcagcatgtatagatcaaccaa | |
| tgtcccaaaaaacaaagtccggcctgagctgcatggg | |
| aacatacggtggaccatctgctcctaacatggtcttc | |
| ggcaaaaatagtacgaatcatcaagctgccaactcga | |
| tcatgatgactatcctggtcacacagagaactgagcc | |
| ggagattcaaaaagcagaactttgggagaaggagttt | |
| ttgaagttctgcaaagagtacagagaaaagtctccga | |
| aagtgattttctcgtttatggcagaaaggtcgatcac | |
| tgatgagattgaaaacgacgcaaaggatgaaattgtg | |
| actgttgtgattgctctggcttttctcatcggttatg | |
| ttacattctctctcgggcgttactttgtgtgtgagaa | |
| tcagctttggtcaattctggtacattctcgaatctgt | |
| cttggaatgctgagcgtcatcataaacttgctcagtt | |
| cgttctgctcgtggggaattttctccatgtttggaat | |
| ccatccggtcaaaaatgcgttggtcgtacaattcttc | |
| gttgtgacgttgctaggcgtctgtcgtacattcatgg | |
| ttgttaagtactacgctcaacaaagagtctccatgcc | |
| ttacatgtcccccgaccaatgtccagagatcgtggga | |
| atggtcatggctggaactatgccagctatgttcagta | |
| gctcgttgggatgtgctttcagctttttcattggtgg | |
| attcacagatttgcctgcaatccgaacattttgtctc | |
| tacgccggtcttgctgttcttattgatgtggtcttac | |
| attgcacaattttccttgcacttttcgtatgggacac | |
| tcaaagggagctcaatggaaaaccagagttctttttc | |
| ccatatcaaatcaaggatcttttgggtgcttacctta | |
| ttggtcgtcagagggcgacagacacttttatgacaca | |
| attcttccactttcaagtagctccgttcctgatgcac | |
| agaatgactcgtattatcacaggaataatcttcattg | |
| cttctttcattacaacagtgatcctctcaagtaagat | |
| cagcgttggattcgaccagagtatggctttcacagag | |
| aaaagctacatcagcacgcactttcgatatctcgaca | |
| aattttttgacgtgggaccgccagtgtttttcactgt | |
| tgacggagaacttgattggcacagaccagatgttcag | |
| aacaagttttgtacatttcctgggtgtagcgacactt | |
| cgtttggaaacattatgaattacgcagttggacacac | |
| ggagcagacgtacttgtccggtgaaatgtataactgg | |
| attgacaactatctggaatggatttcaagaaaaagcc | |
| catgttgtaaagtttatgtgcacgaccccaacacatt | |
| ctgctcgacaaaccggaacaagagtgcgttagacgat | |
| aaggcatgtagaacttgcatggactttgattacgtgg | |
| ccaactcgtacccgaaaagctcaatcatgtaccatcg | |
| tccgtcaatcgaagttttctaccgtcatctgagacac | |
| tttttagaagacactccaaattctgaatgcgttttcg | |
| gtggccgtgcttcattcaaagatgcaatcagcttcac | |
| atctagaggacgtattcaagcatctcaattcatgact | |
| ttccacaaaaagctgtcaattagcaactccagtgatt | |
| tcatcaaggcaatggacactgctcgaatggtatcccg | |
| tcgccttgaaagatcaattgacgatacagctcacgtc | |
| ttcgcatacagcaaaattttcccattctacgaacagt | |
| actctactatcatgccaattctcactacccaactttt | |
| tatcactgttgttggcgtcttcggcatcatttgtgtt | |
| actcttgggattgatgtgaaaggagcggcttgcgctg | |
| taatttgtcaagtgtccaactacttccatattgtggc | |
| atttatgtatatctttaatatccccgtcaatgccttg | |
| tctgcaacaaatcttgtaatgtcgtctggaattctaa | |
| tcgagttttctgtaaatgttctcaaaggatacgcctg | |
| ctcacttcgccagcgagcaaaagatcgcgcagagtct | |
| acggttggaagtattggaccaatcattctctccggcc | |
| cagttgtcactatggctggatctacaatgtttctctc | |
| gggggctcatcttcagattataacggtctactttttc | |
| aaattgttcctcatcactattgtcagcagtgctgtcc | |
| atgctctcatcatattgcctattttgcttgccttcgg | |
| aggatcgagaggccatggaagttcagagactagcacg | |
| aatgacaatgacgagcaacatgacgcgtgcgttttgt | |
| cgccaaccgcggaaagtcatatttcaaacgtggaaga | |
| aggaatccttaaccgtccttctttactggacgccagc | |
| cacattctcgatccgcttcttaaagcggaaggtggga | |
| ttgataaagcaattgatatcataacaatcgatcgctc | |
| ttacccatctacgccatcttcactgccatgtacctcc | |
| cgaatgccaagagctcatatagaaccggatctccgat | |
| ctttgtag | |

ncr-1 Protein:

| | |
|---|---|
| MKQLLIFCLL FGSIFHHGDA GCIMRGLCQK | (SEQ ID NO:2) |
| HTENAYGPCV TNDTNVEPTA FDKTHPAYEK | |
| MVEFCPHLLT GDNKLCCTPS QAEGLTKQIA | |
| QARHILGRCP SCFDNFAKLW CEFTCSPNQQ | |
| DFVSISEMKP IEKKEGFTPE YQPAEAYVNT | |
| VEYRLSTDFA EGMFSSCKDV TFGGQPALRV | |
| MCTSTPCTLT NWLEFIGTQN LDLNIPIHTK | |
| FLLYDPIKTP PSDRSTYMNV NFTGCDKSAR | |
| VGWPACSTSE CNKEEYANLI DLDDGKTSGQ | |
| TCNVHGIACL NIFVMLAFIG SLAVLLCVGF | |
| VFTSYDEDYT NLRQTQSGEE SPKRNRIKRT | |
| GAWIHNFMEN NARDIGMMAG RNPKSHFFIG | |
| CAVLIFCLPG MIYHKESTNV VDMWSSPRSR | |
| ARQEEMVFNA NFGRPQRYQQ IMLLSHRDFQ | |
| SSGKLYGPVF HKDIFEELFD ILNAIKNIST | |
| QDSDGRTITL DDVCYRPMGP GYDCLIMSPT | |
| NYFQGNKEHL DMKSNKEETV SEDDDAFDYF | |
| SSEATTDEWM NHMAACIDQP MSQKTKSGLS | |
| CMGTYGGPSA PNMVFGKNST NHQAANSIMM | |
| TILVTQRTEP EIQKAELWEK EFLKFCKEYR | |
| EKSPKVIFSF MAERSITDEI ENDAKDEIVT | |
| VVIALAFLIG YVTFSLGRYF VCENQLWSIL | |
| VHSRICLGML SVIINLLSSF CSWGIFSMFG | |
| IHPVKNALVV QFFVVTLLGV CRTFMVVKYY | |
| AQQRVSMPYM SPDQCPEIVG MVMAGTMPAM | |
| FSSSLGCAFS FFIGGFTDLP AIRTFCLYAG | |
| LAVLIDVVLH CTIFLALFVW DTQRELNGKP | |
| EFFFPYQIKD LLGAYLIGRQ RATDTFMTQF | |
| FHFQVAPFLM HRMTRIITGI IFIASFITTV | |
| ILSSKISVGF DQSMAFTEKS YISTHFRYLD | |
| KFFDVGPPVF FTVDGELDWH RPDVQNKFCT | |
| FPGCSDTSFG NIMNYAVGHT EQTYLSGEMY | |
| NWIDNYLEWI SRKSPCCKVY VHDPNTFCST | |
| NRNKSALDDK ACRTCMDFDY VANSYPKSSI | |
| MYHRPSIEVF YRHLRHFLED TPNSECVFGG | |
| RASFKDAISF TSRGRIQASQ FMTFHKKLSI | |
| SNSSDFIKAM DTARMVSRRL ERSIDDTAHV | |
| FAYSKIFPFY EQYSTIMPIL TTQLFITVVG | |
| VFGIICVTLG IDVKGAACAV ICQVSNYFHI | |
| VAFMYIFNIP VNALSATNLV MSSGILIEFS | |
| VNVLKGYACS LRQRAKDRAE STVGSIGPII | |
| LSGPVVTMAG STMFLSGAHL QIITVYFFKL | |
| FLITIVSSAV HALIILPILL AFGGSRGHGS | |
| SETSTNDNDE QHDACVLSPT AESHISNVEE | |
| GILNRPSLLD ASHILDPLLK AEGGIDKAID | |
| IITIDRSYPS TPSSLPCTSR MPRAHIEPDL | |
| RSL | |

ncr-2Spliced Coding Region (Genbank Accession No. F09G8.4):

| | |
|---|---|
| atgcgtcaaggaggaggaggaggcgagagaatggtat | (SEQ ID NO:3) |
| ctgtcctattcttattgctaatacatttggcattgtg | |
| ccaagcaaaatgtgtgatgacggaatgtgacggagag | |
| gaggatagcaaccatccaccatgcaagactaacaagt | |
| caacatatctaccaatcaccgtgacacggtctctaaa | |
| tccaacttatatggctcgattcgaaaagtactgctcg | |
| tatcttgtacaggaagaggataaagctcaagtctgct | |
| gtacagaactgcaattaaaaggaatgactgatcgaat | |
| ttctaatgctgctacaatccttggatcttgtcccagt | |
| tgcttcgacaattttgcaaaactctggtgtcaattca | |
| cttgttccccagatcaatcaaaattcatgaaggtaat | |
| ggaaacgactggtccgaaaaatgtcgtggttaaaatg | |
| gagttcaaagtgaatcgagattttgtggagggattgt | |
| acgagtcatgccgtcatacgtggtttgccaatggact | |
| tgctcttcgattgatgtctcttggtggaaaagtctct | |
| tttgaaaacttttatggatttatgggaacgaagaatc | |
| ttgcacagagtattccaataaacaccgagtttcagtt | |
| ttctcgaatgaaaaacgcaatgaatattccaacaaca | |
| ccatgtcacaaatcagctggccccaaagttccagctt | |
| gtggagctattgattgtccaacaaatgcccatcaact | |
| tgtagatatctccaaagttgaacatctcggaacgaaa | |
| gtgttccatcctcacttcccagactttgaatggttac | |
| tgaaaatctgcggatgcctcgcacttacagtcctcct | |
| ggtcttcattcttaaatattcttgtcatagacgatca | |
| gcgcctaatggtgaagatggatgttacgtggatttgg | |
| gaaaaggaaatttggaagttcaatttgaaggactttg | |
| tgctcgatatgcaaatgctgtaattaaacatccattg | |
| atatttgtttcacttggacttattgtagctgcagctt | |
| gttgctctggaaatttcaaatttcacagtctcacgca | |
| ttcagttgatcaagtttcggctgccgatggagaaact | |
| agacggaacgagaaaaaattcatccattctttcggac | |
| ctaatcatcgaattgagcaaatattcatcaacttacc | |
| acctaccactaagtctatgttcaacatgccacttttt | |
| gaagaaatgttccaattggttgggaatattcagaatt | |
| tgacagcttgctatgggaattcgtcagtgaagcttga | |
| tgatatatgctacaaacctatcggaaaaaatcacggg | |
| tgtgcaattatgagcccaaccaactatttccagaaca | |
| agtggaccaatttcgaaaacgcgggacctccaactat | |
| tgatgatgagatctttgatgatcaacactgggaacat | |
| ttgaagtactgtataagaaatcctcttaccgtatcca | |
| catactctgaaatgtcatgtttcggagagttttctgg | |
| gcctatcgatccgattctagtgtttggtggaagtaat | |
| gaaagtatcaagggtgctgaaatgtactacaccgctc | |
| ggactattatgataacggttttaatcagaggcccaga | |
| agatcaagctatagcctgggaaactgcgtttctaaac | |
| atgatgagtcgttatgaaatgaaacacgcaaatttta | |
| cattcatgacagaaacgtctgttgcggaagaaattca | |
| tactgctgtggagactgataaaattgtgtctgtgatt | |
| gcatgtgctgctgttcttatatgggttattacaatgc | |
| ttggaattaatcattggccagagtcatccatattgtc | |
| agctctagttcatcacaagctactcatttcgatatca | |
| gctgttatgatcagtgtgatcagtgtttggtgctcga | |
| ttggtatgttctcgctctttggtgtccatgcaacaga | |
| taatgcaattgttgttttattctttgtcatcacttgt | |
| cttggaatcaatcgaatctttgtcattatcagaacat | |
| ttcaagccaatggtcactgctacggacttccgaatat | |
| ctcatatcgagaaatgaatcacagaatttcaaatgta | |
| atgcgtcgaagtattccaattgttctaacaaactccc | |
| ttatctgttcaacatgccttttcttagccggaggtgt | |
| tcttccatatgtatctgtcagcatgccagcagttgaa | |
| gtatttgcaagacatgctgggttggcaattcttatgg | |
| atactgcattctatctccttgtaatgctaccactatt | |
| tcaatatgatgcgagaagagaaatgtctggaaaatgt | |
| gaaatctggccgtggtacgagctatcaaacgagtcaa | |
| aaattaatctatgcatggaagcagttgatggaaatct | |
| tcgaagcccagtggattggttcaagcttgcgatcgcc | |
| cctttattgctcaaaaagatttgtagaatttggatag | |
| ctacattcttctttgtttccctaattatagcatgcta | |
| ctgtactctgtgcctcgaatttggtttcaatcaagtt | |
| atggctttttcagagacaagttatctgacaaaacatt | |
| tccaaaatatgaatgagaatttgaacattgggcctcc | |
| gttatggtttgttgtagaaggtgatgtcaaatggcat | |
| gatccgaaaatgcagaacaagttttgtacattagccg | |
| gatgtgatgataattcaatgggaaataagatccgatc | |
| attggcatacgctgagaactacaaaggaaactatcta | |
| cacggcgatgtaaatatttggctggattcatatcttc | |
| agttcatgcatcctcgaggttcatgctgcaaaatgga | |
| tggaaaacaattttgtgacccatcaaacgcaacacat | |
| tgctcatcttgctcgtcgagcagcgtagcttcattga | |
| caactacagaatatgagttctaccgtaatttgcatca | |
| cttcttggagactcctccatcaatccaatgtgctcat | |
| ggtggaatggctctggcaaaaccagcgatcaatttga | |
| caagaaatggaaaaattcaatctgcctatttttcaac | |
| atttttcaaaaagttgaatctctcggactctattcag | |
| ctgtacgatgcctggagattcgcgaaatacctagcag | |
| acgacattgaacgtgaactggaaattcctggtgtgaa | |
| agtctacgtgtacagtaccttcttcccttattacgaa | |
| caatatctgactctgagcacaactgtatatactcttg | |
| tagttctggttctttttgttgctttcgtcacgatttc | |
| cctatttttacgcgttaatttggctggaagtttagtc | |
| actgtatttgtactcctctcatcatatcttcacttga | |
| tggaatggatgtatctactcggaatcacagtgaatgt | |
| agtatcagttatcaatatggcaatgtctcttggaatc | |
| gctgttgagttttttgggcaaatgctacatggattct | |
| ataactcaaaaaaaccgaaaagggaagaaagagcatt | |
| cgctgcacttgtcagtaatggatctacgacattgtcc | |
| ggaatcttccctgctatcatgatcaccgctggatgtc | |
| tttcatttgctgattctcgtgtgctcatcacatactt | |
| ctgcaatcaacttgttggaattggtcttgtttgtgca | |
| gttcatggagttgtttacatgccaacacttttggcaa | |
| ttttcggatcggatttctatcagaatgtgagcagtga | |
| agaagaatcaaccgatgaagcagaactgcaggatact | |
| cctccatcgaccacatcaagtacttccagcacttctg | |
| aaacatctgtctga | |

ncr-2 Protein:

| | |
|---|---|
| MRQGGGGGER MVSVLFLLLI HLALCQAKCV | (SEQ ID NO:4) |
| MTECDGEEDS NHPPCKTNKS TYLPITVTRS | |
| LNPTYMARFE KYCSYLVQEE DKAQVCCTEL | |
| QLKGMTDRIS NAATILGSCP SCFDNFAKLW | |
| CQFTCSPDQS KFMKVMETTG PKNVVVKMEF | |
| KVNRDFVEGL YESCRHTWFA NGLALRLMSL | |
| GGKVSFENFY GFMGTKNLAQ SIPINTEFQF | |
| SRMKNAMNIP TTPCHKSAGP KVPACGAIDC | |
| PTNAHQLVDI SKVEHLGTKV FHPHFPDFEW | |
| LLKICGCLAL TVLLVFILKY SCHRRSAPNG | |
| EDGCYVDLGK GNLEVQFEGL CARYANAVIK | |
| HPLIFVSLGL IVAAACCSGN FKFHSLTHSV | |
| DQVSAADGET RRNEKKFIHS FGPNHRIEQI | |
| FINLPPTTKS MFNMPLFEEM FQLVGNIQNL | |
| TACYGNSSVK LDDICYKPIG KNHGCAIMSP | |
| TNYFQNKWTN FENAGPPTID DEIFDDQHWE | |
| HLKYCIRNPL TVSTYSEMSC FGEFSGPIDP | |
| ILVFGGSNES IKGAEMYYTA RTIMITVLIR | |
| GPEDQAIAWE TAFLNMMSRY EMKHANFTFM | |
| TETSVAEEIH TAVETDKIVS VIACAAVLIW | |
| VITMLGINHW PESSILSALV HHKLLISISA | |
| VMISVISVWC SIGMFSLFGV HATDNAIVVL | |
| FFVITCLGIN RIFVIIRTFQ ANGHCYGLPN | |
| ISYREMNHRI SNVMRRSIPI VLTNSLICST | |
| CLFLAGGVLP YVSVSMPAVE VFARHAGLAI | |
| LMDTAFYLLV MLPLFQYDAR REMSGKCEIW | |
| PWYELSNESK INLCMEAVDG NLRSPVDWFK | |
| LAIAPLLLKK ICRIWIATFF FVSLIIACYC | |
| TLCLEFGFNQ VMAFSETSYL TKHFQNMNEN | |
| LNIGPPLWFV VEGDVKWHDP KMQNKFCTLA | |
| GCDDNSMGNK IRSLAYAENY KGNYLHGDVN | |
| IWLDSYLQFM HPRGSCCKMD GKQFCDPSNA | |
| THCSSCSSSS VASLTTTEYE FYRNLHHFLE | |
| TPPSIQCAHG GMALAKPAIN LTRNGKIQSA | |
| YFSTFFKKLN LSDSIQLYDA WRFAKYLADD | |
| IERELEIPGV KVYVYSTFFP YYEQYLTLST | |
| TVYTLVVLVL FVAFVTISLF LRVNLAGSLV | |
| TVFVLLSSYL HLMEWMYLLG ITVNVVSVIN | |
| MAMSLGIAVE FFGQMLHGFY NSKKPKREER | |
| AFAALVSNGS TTLSGIFPAI MITAGCLSFA | |
| DSRVLITYFC NQLVGIGLVC AVHGVVYMPT | |
| LLAIFGSDFY QNVSSEEEST DEAELQDTPP | |
| STTSSTSSTS ETSV | |

In an embodiment of the invention, the human NPC1L1 gene comprises the following nucleotide sequence:

| | |
|---|---|
| ATGGCGGAGGCCGGCCTGAGGGGCTGGCTGCTGTGGG | (SEQ ID NO:5) |
| CCCTGCTCCTGCGCTTGGCCCAGAGTGAGCCTTACAC | |
| AACCATCCACCAGCCTGGCTACTGCGCCTTCTATGAC | |
| GAATGTGGGAAGAACCCAGAGCTGTCTGGAAGCCTCA | |
| TGACACTCTCCAACGTGTCCTGCCTGTCCAACACGCC | |
| GGCCCGCAAGATCACAGGTGATCACCTGATCCTATTA | |
| CAGAAGATCTGCCCCCGCCTCTACACCGGCCCCAACA | |
| CCCAAGCCTGCTGCTCCGCCAAGCAGCTGGTATCACT | |
| GGAAGCGAGTCTGTCGATCACCAAGGCCCTCCTCACC | |
| CGCTGCCCAGCCTGCTCTGACAATTTTGTGAACCTGC | |
| ACTGCCACAACACGTGCAGCCCCAATCAGAGCCTCTT | |
| CATCAATGTGACCCGCGTGGCCCAGCTAGGGGCTGGA | |
| CAACTCCCAGCTGTGGTGGCCTATGAGGCCTTCTACC | |
| AGCATAGCTTTGCCGAGCAGAGCTATGACTCCTGCAG | |
| CCGTGTGCGCGTCCCTGCAGCTGCCACGCTGGCTGTG | |
| GGCACCATGTGTGGCGTGTATGGCTCTGCCCTTTGCA | |
| ATGCCCAGCGCTGGCTCAACTTCCAGGGAGACACAGG | |
| CAATGGTCTGGCCCCACTGGACATCACCTTCCACCTC | |
| TTGGAGCCTGGCCAGGCCGTGGGGAGTGGGATTCAGC | |
| CTCTGAATGAGGGGGTTGCACGTTGCAATGAGTCCCA | |
| AGGTGACGACGTGGCGACCTGCTCCTGCCAAGACTGT | |
| GCTGCATCCTGTCCTGCCATAGCCCGCCCCCAGGCCC | |
| TCGACTCCACCTTCTACCTGGGCCAGATGCCGGGCAG | |
| TCTGGTCCTCATCATCATCCTCTGCTCTGTCTTCGCT | |
| GTGGTCACCATCCTGCTTGTGGGATTCCGTGTGGCCC | |
| CCGCCAGGGACAAAAGCAAGATGGTGGACCCCAAGAA | |
| GGGCACCAGCCTCTCTGACAAGCTCAGCTTCTCCACC | |
| CACACCCTCCTTGGCCAGTTCTTCCAGGGCTGGGGCA | |
| CGTGGGTGGCTTCGTGGCCTCTGACCATCTTGGTGCT | |
| ATCTGTCATCCCGGTGGTGGCCTTGGCAGCGGGCCTG | |
| GTCTTTACAGAACTCACTACGGACCCCGTGGAGCTGT | |
| GGTCGGCCCCCAACAGCCAAGCCCGGAGTGAGAAAGC | |
| TTTCCATGACCAGCATTTCGGCCCCTTCTTCCGAACC | |
| AACCAGGTGATCCTGACGGCTCCTAACCGGTCCAGCT | |
| ACAGGTATGACTCTCTGCTGCTGGGGCCCAAGAACTT | |
| CAGCGGAATCCTGGACCTGGACTTGCTGCTGGAGCTG | |
| CTAGAGCTGCAGGAGAGGCTGCGGCACCTCCAGGTAT | |
| GGTCGCCCGAAGCACAGCGCAACATCTCCCTGCAGGA | |
| CATCTGCTACGCCCCCCTCAATCCGGACAATACCAGT | |
| CTCTACGACTGCTGCATCAACAGCCTCCTGCAGTATT | |
| TCCAGAACAACCGCACGCTCCTGCTGCTCACAGCCAA | |
| CCAGACACTGATGGGGCAGACCTCCCAAGTCGACTGG | |
| AAGGACCATTTTCTGTACTGTGCCAATGCCCCGCTCA | |
| CCTTCAAGGATGGCACAGCCCTGGCCCTGAGCTGCAT | |
| GGCTGACTACGGGGCCCCTGTCTTCCCCTTCCTTGCC | |
| ATTGGGGGGTACAAAGGAAAGGACTATTCTGAGGCAG | |
| AGGCCCTGATCATGACGTTCTCCCTCAACAATTACCC | |
| TGCCGGGGACCCCCGTCTGGCCCAGGCCAAGCTGTGG | |
| GAGGAGGCCTTCTTAGAGGAAATGCGAGCCTTCCAGC | |
| GTCGGATGGCTGGCATGTTCCAGGTCACGTTCACGGC | |
| TGAGCGCTCTCTGGAAGACGAGATCAATCGCACCACA | |
| GCTGAAGACCTGCCCATCTTTGCCACCAGCTACATTG | |
| TCATATTCCTGTACATCTCTCTGGCCCTGGGCAGCTA | |
| TTCCAGCTGGAGCCGAGTGATGGTGGACTCCAAGGCC | |
| ACGCTGGGCCTCGGCGGGGTGGCCGTGGTCCTGGGAG | |
| CAGTCATGGCTGCCATGGGCTTCTTCTCCTACTTGGG | |
| TATCCGCTCCTCCCTGGTCATCCTGCAAGTGGTTCCT | |
| TTCCTGGTGCTGTCCGTGGGGGCTGATAACATCTTCA | |
| TCTTTGTTCTCGAGTACCAGAGGCTGCCCCGGAGGCC | |
| TGGGGAGCCACGAGAGGTCCACATTGGGCGAGCCCTA | |
| GGCAGGGTGGCTCCCAGCATGCTGTTGTGCAGCCTCT | |
| CTGAGGCCATCTGCTTCTTCCTAGGGGCCCTGACCCC | |
| CATGCCAGCTGTGCGGACCTTTGCCCTGACCTCTGGC | |
| CTTGCAGTGATCCTTGACTTCCTCCTGCAGATGTCAG | |
| CCTTTGTGGCCCTGCTCTCCCTGGACAGCAAGAGGCA | |
| GGAGGCCTCCCGGTTGGACGTCTGCTGCTGTGTCAAG | |
| CCCCAGGAGCTGCCCCCGCCTGGCCAGGGAGAGGGGC | |
| TCCTGCTTGGCTTCTTCCAAAAGGCTTATGCCCCCTT | |
| CCTGCTGCACTGGATCACTCGAGGTGTTGTGCTGCTG | |
| CTGTTTCTCGCCCTGTTCGGAGTGAGCCTCTACTCCA | |
| TGTGCCACATCAGCGTGGGACTGGACCAGGAGCTGGC | |
| CCTGCCCAAGGACTCGTACCTGCTTGACTATTTCCTC | |
| TTTCTGAACCGCTACTTCGAGGTGGGGGCCCCGGTGT | |
| ACTTTGTTACCACCTTGGGCTACAACTTCTCCAGCGA | |
| GGCTGGGATGAATGCCATCTGCTCCAGTGCAGGCTGC | |
| AACAACTTCTCCTTCACCCAGAAGATCCAGTATGCCA | |
| CAGAGTTCCCTGAGCAGTCTTACCTGGCCATCCCTGC | |
| CTCCTCCTGGGTGGATGACTTCATTGACTGGCTGACC | |
| CCGTCCTCCTGCTGCCGCCTTTATATATCTGGCCCCA | |
| ATAAGGACAAGTTCTGCCCCTCGACCGTCAACTCTCT | |
| GAACTGCCTAAAGAACTGCATGAGCATCACGATGGGC | |
| TCTGTGAGGCCCTCGGTGGAGCAGTTCCATAAGTATC | |
| TTCCCTGGTTCCTGAACGACCGGCCCAACATCAAATG | |
| TCCCAAAGGCGGCCTGGCAGCATACAGCACCTCTGTG | |
| AACTTGACTTCAGATGGCCAGGTTTTAGCCTCCAGGT | |
| TCATGGCCTATCACAAGCCCCTGAAAAACTCACAGGA | |
| TTACACAGAAGCTCTGCGGGCAGCTCGAGAGCTGGCA | |
| GCCAACATCACTGCTGACCTGCGGAAAGTGCCTGGAA | |
| CAGACCCGGCTTTTGAGGTCTTCCCCTACACGATCAC | |
| CAATGTGTTTTATGAGCAGTACCTGACCATCCTCCCT | |
| GAGGGGCTCTTCATGCTCAGCCTCTGCCTTGTGCCCA | |
| CCTTCGCTGTCTCCTGCCTCCTGCTGGGCCTGGACCT | |
| GCGCTCCGGCCTCCTCAACCTGCTCTCCATTGTCATG | |
| ATCCTCGTGGACACTGTCGGCTTCATGGCCCTGTGGG | |
| ACATCAGTTACAATGCTGTGTCCCTCATCAACCTGGT | |
| CTCGGCGGTGGGCATGTCTGTGGAGTTTGTGTCCCAC | |
| ATTACCCGCTCCTTTGCCATCAGCACCAAGCCCACCT | |
| GGCTGGAGAGGGCCAAAGAGGCCACCATCTCTATGGG | |
| AAGTGCGGTGTTTGCAGGTGTGGCCATGACCAACCTG | |
| CCTGGCATCCTTGTCCTGGGCCTCGCCAAGGCCCAGC | |
| TCATTCAGATCTTCTTCTTCCGCCTCAACCTCCTGAT | |
| CACTCTGCTGGGCCTGCTGCATGGCTTGGTCTTCCTG | |
| CCCGTCATCCTCAGCTACGTGGGGCCTGACGTTAACC | |
| CGGCTCTGGCACTGGAGCAGAAGCGGGCTGAGGAGGC | |
| GGTGGCAGCAGTCATGGTGGCCTCTTGCCCAAATCAC | |
| CCCTCCCGAGTCTCCACAGCTGACAACATCTATGTCA | |
| ACCACAGCTTTGAAGGTTCTATCAAAGGTGCTGGTGC | |
| CATCAGCAACTTCTTGCCCAACAATGGGCGGCAGTTC | |
| TGA | |

In an embodiment of the invention, the human NPC1L1 protein comprises the following amino acid sequence:

| | |
|---|---|
| MAEAGLRGWL LWALLLRLAQ SEPYTTIHQP | (SEQ ID NO:6) |
| GYCAFYDECG KNPELSGSLM TLSNVSCLSN | |
| TPARKITGDH LILLQKICPR LYTGPNTQAC | |
| CSAKQLVSLE ASLSITKALL TRCPACSDNF | |
| VNLHCHNTCS PNQSLFINVT RVAQLGAGQL | |
| PAVVAYEAFY QHSFAEQSYD SCSRVRVPAA | |
| ATLAVGTMCG VYGSALCNAQ RWLNFQGDTG | |
| NGLAPLDITF HLLEPGQAVG SGIQPLNEGV | |
| ARCNESQGDD VATCSCQDCA ASCPAIARPQ | |
| ALDSTFYLGQ MPGSLVLIII LCSVFAVVTI | |
| LLVGFRVAPA RDKSKMVDPK KGTSLSDKLS | |
| FSTHTLLGQF FQGWGTWVAS WPLTILVLSV | |
| IPVVALAAGL VFTELTTDPV ELWSAPNSQA | |
| RSEKAFHDQH FGPFFRTNQV ILTAPNRSSY | |
| RYDSLLLGPK NFSGILDLDL LLELLELQER | |
| LRHLQVWSPE AQRNISLQDI CYAPLNPDNT | |
| SLYDCCINSL LQYFQNNRTL LLLTANQTLM | |
| GQTSQVDWKD HFLYCANAPL TFKDGTALAL | |
| SCMADYGAPV FPFLAIGGYK GKDYSEAEAL | |
| IMTFSLNNYP AGDPRLAQAK LWEEAFLEEM | |
| RAFQRRMAGM FQVTFTAERS LEDEINRTTA | |
| EDLPIFATSY IVIFLYISLA LGSYSSWSRV | |
| MVDSKATLGL GGVAVVLGAV MAAMGFFSYL | |
| GIRSSLVILQ VVPFLVLSVG ADNIFIFVLE | |
| YQRLPRRPGE PREVHIGRAL GRVAPSMLLC | |
| SLSEAICFFL GALTPMPAVR TFALTSGLAV | |
| ILDFLLQMSA FVALLSLDSK RQEASRLDVC | |
| CCVKPQELPP PGQGEGLLLG FFQKAYAPFL | |
| LHWITRGVVL LLFLALFGVS LYSMCHISVG | |
| LDQELALPKD SYLLDYFLFL NRYFEVGAPV | |
| YFVTTLGYNF SSEAGMNAIC SSAGCNNFSF | |
| TQKIQYATEF PEQSYLAIPA SSWVDDFIDW | |
| LTPSSCCRLY ISGPNKDKFC PSTVNSLNCL | |
| KNCMSITMGS VRPSVEQFHK YLPWFLNDRP | |
| NIKCPKGGLA AYSTSVNLTS DGQVLASRFM | |
| AYHKPLKNSQ DYTEALRAAR ELAANITADL | |
| RKVPGTDPAF EVFPYTITNV FYEQYLTILP | |
| EGLFMLSLCL VPTFAVSCLL LGLDLRSGLL | |
| NLLSIVMILV DTVGFMALWD ISYNAVSLIN | |
| LVSAVGMSVE FVSHITRSFA ISTKPTWLER | |
| AKEATISMGS AVFAGVAMTN LPGILVLGLA | |
| KAQLIQIFFF RLNLLITLLG LLHGLVFLPV | |
| ILSYVGPDVN PALALEQKRA EEAVAAVMVA | |
| SCPNHPSRVS TADNIYVNHS FEGSIKGAGA | |
| ISNFLPNNGR QF | |

Human NPC1L1 is also disclosed at Genbank Accession Nos. NP_{—}037521 and AF192522 and at Davies, et al., Genomics 65(2):137-45 (2000) and Ioannou, Mol. Genet. Metab. 71(1-2):175-81 (2000); see also Published U.S. Patent Application No. 2004/0161838.

In an embodiment of the invention, the vit-2 promoter comprises the following nucleotide sequence:

(SEQ ID NO:7)

cctgtgttgcggattcgccaagtgcgcagcgaccacc
ccagcggcgttgtcgatttcaacaactagcgtgatattggttt
tcgtggcataaagtacttgatcaaaaatagtcgactttctgaa
cgggaagtaaaaaaaaaatacaaacaaaacagatttcttataa
gttacaaataaataatggtgaattcaagttaatttagaaacaa
ttaacacacaatcacagtaccggacgtttgtgagaccttatag
tccagatttgggaattactgacaaccgaataatacgcagaaca
ctggaaaatgaaaaagttttagggatgccgcggatttccaact
aatcagatttttgctagacgtacaattaatgttaagttagaat
aatgtcaaaacatctggtgaagtttgtgaataattgggttttt
aatgtttagttgttttaagttttaacatttgttagttctatca
tatttttttggattcagttttagtttttgtgatcctgtgtgcc
caattgaggaaaactggttgcgttttaggtgcctacaatacaa
ttttaccaagttttttttgcagatttttctatcctgtcggtca
caatgcaactagcaagtaaaaaagtaatgactttgttttaaat
accatgtgctagctgagtttcatcatgtccagaatcacctcat
cgttaaaaagtcatgagtatcaaagtgcagtgtgatcaaactg
tattacttgaaacaatttagttatatgtttagaacccctcatt
caaaattaatagacagggctctcaccgaatgttgcaatttgtt
tctgataagggtcacaaagcggagcgaatgcttgaatgtgtcc
atcaatgagcttatcaatgcgctaaaacgctataacttccata
tgaagtcaatcgaacatatgtcaatctttagccgtatataaag
gtgcactgaaaacagtccaatcacggttcagc

The vit-2 promoter sequence is also available under Genbank Accession No. U56966.

In an embodiment of the invention, the col-19 promoter comprises the following nucleotide sequence:

| | |
|---|---|
| ttagtaatctcatacatataaagatgacattttccta | (SEQ ID NO:8) |
| catatgtataaaaattgcaaaaagtgaaacttcaaat | |
| atccatattctaagactttttttcaatctgctccaat | |
| ttctcaaatctgtttaaatttttgacaaatgttaatc | |
| agttttcggctatttttgatgtcacttggtcttagag | |
| cttatatagcttaacagtcaaaaaagttacgcacttg | |
| gaacaaaaaatgctctaaaatttccagataagaattg | |
| gaaaggttttgatttgttcctttcatgaattgtggtt | |
| ttcaattgtttgttttaaaactattttcaaaaatgca | |
| gcttgcgatcattcatgtgttgttcgtagtttgtgac | |
| gtcaattttaaaatcttcaagaaatacttgcatttct | |
| ataaaattgatttgaaattttgaagcctaaattactg | |
| tcaaaaattttgattcgaaggttttctaaaacaatga | |
| gtgatttcgggcgttatatcaaatatccaaatatata | |
| aaatttcataaaactactataaactatatgctagaaa | |
| ctcgtatttatactagaaatactagaaactagaaact | |
| ataaaaacctggaacatcaaataaatttaatgtcaga | |
| ctacaaccctttaaactacataccaacgatttattag | |
| tttgagcttcaagaattttatattcttaagagggacg | |
| cacaaaaaatcactcttcacactatttctttgttgaa | |
| tagaggtcagcggcttttaaaaattataatttgaaaa | |
| aaaattgggactttttcaaaaaaaaagcttccaaacg | |
| tccctattaggaaatgtaaacttattcccaaaaaatt | |
| aaaaattccagagaaagtagacaaatttcagaaaact | |
| taccgcgctacatctaatactttctcataggtttttt | |
| tattgggaaactgatgaaaattatttgaattcataat | |
| aaaataaattcgtatttagcatttgaaaatttgcacc | |
| aatgtattatttaattttttttttcgaaaatttaacg | |
| cattttctctctaaaaactcgaaatttagtgtgttct | |
| aaacaacagtaagcatacaatacctttgttcaaaatt | |
| gacgtgctttctgaaccaatatggttagtttcaaaaa | |
| tttttgtattataggatagaaatatttggaaataaat | |
| tttaaaaccaaacttatgcctttctcttttagtatcc | |
| cagctaggtaatattttagtatttgcccaaatccttg | |
| aagtaaggagtatataatttttgaaaaacaataaaac | |
| tccagataattcatagttttttctcgaaagaaaattt | |
| ttgagattagttattgaacttcatttttgaacattat | |
| tcgttgaaaaacactcgctttgtctttattttcaaaa | |
| aaattccgatttccccaaccagaaaaaaaaacagata | |
| gaagaaatttctccttaaatttcattgtccatctctc | |
| ttggaaacacattatctatcaaatgaaaaacgcattt | |
| tttttcctggcagaaaaatgaaattggttagattaca | |
| ctggttaggtttgaaggtgtaactttcgctttctcag | |
| caactttcagtataaaaggaaacggtcaccatttaga | |
| aagacatcagttcat | |

The col-19 promoter sequence is also available under Genbank Accession No. Genbank U41553.

In an embodiment of the invention, the elt-2 promoter comprises the following nucleotide sequence:

| | |
|---|---|
| gatcttctccttcccatgtgctgcaaaccacaacttc | (SEQ ID NO:9) |
| cggcagccacggaggtatgtgataagttgaatgagtc | |
| atcatgacgtttgaaaaattacaacgaattgattaat | |
| cgacaaatttcagcagattcaaccatttcgtcaacaa | |
| catctttggcatcacctgtcactttaccaatgactcc | |
| atcttcagattcttctactccaatgcacgccatcgaa | |
| cccgttcagcatctctaatttcgaaatgtatgaactc | |
| caattcttgataaccaattcctgactctgtaactatt | |
| attattataatcctattacccaccttttttcccattc | |
| tgctcttagttctcccccaacccgtacattttctcaa | |
| ctaataaagacaatgagaaagtgagagagaataagac | |
| gaactgaaaagaatgtgtaaacagtttattgcctctc | |
| gattgtaccatctagtgtcttgtcgtgtgcataacta | |
| ctgataacagatcgaatgtgaaagacccaataaatgc | |
| ttggaaagtatcaaatatgtttagaaatagtatttag | |
| agacttatgtgggacgaaaaaacaggagcattaagaa | |
| tacagttgcaaacattcatcaagttataatctaataa | |
| tataaattcaaaaatgttaatgataaaaaatgatgat | |
| caaagtaggaaattagtggcgagaaaacaataaatca | |
| tcagaaaatgactgatcttgagtagagtgcttttttt | |
| gtagatttctgattctaaaactgaaaaaaaaactaaa | |
| aatgctcgatgagtcatactaattgaaaaataatcag | |
| attgaaaacgttaaaaatgtacttgctaacatacttc | |
| catggttatttttgaacacaaataaagttatatacat | |
| ctttaccggcaccagaagaaatcaccgctaaccaatg | |
| ttcagtttttacctgaatatccctgaaagtacaaaaa | |
| aatcaatgttcaatcaagtttttgcatgccttcgtgt | |
| gatggctcgcaccaagtattctgacagttataaaatg | |
| tttcctgtgatcatgaatttgaaaaaaaaagaactga | |
| tgggaaaatgtgatattcataaatgaaatgtttaata | |
| aacaaaaaagaaccgtcatcacaaatctactctgaaa | |
| cgttcacagttcctaattgaccaattcagaatttttt | |
| gtactgacttaataaatataaatttacagttttaaga | |
| aacataaattgaattatataaataaaaaaaaaaaaac | |
| taaaatgaagacgtgatggaatgtttcacgtattaaa | |
| aaagctctcggaccgtttgacatccagaaattatgtg | |
| aaagtgattttctaaccctttgacgtcaaaaaagttt | |
| ttcttttttgaaagatacgctgtttagaaatacttat | |
| acagctgattttagaatctccaaaaaaccttcaatta | |
| cttaacaaactttctaaataaagaattttgattttga | |
| ttttctttgaacttcaaaccccccgttaaaaaaccaa | |
| attaaaaaaaagataccagattaattttaaagtgaaa | |
| aatataaatttattcccgactgttaaaaacggtttat | |
| aaacggagtcacaacactcatatacaactatcgacac | |
| caaataaataacttcgaaagctaattgtgtcatttcg | |
| gatatagagagtagtgtgagcagatgtgtttttgcag | |
| aactaaattgatgacactattatggaatataatgacc | |
| aaatgttaaatgtgttaaggtttgatatcaaaacctg | |
| tattttctttttatacagaattgataatgttatcttc | |
| aattgatttctacttctgagctacggcgatacgagga | |
| cgcattctcaacgataatgttgccattttgtcctgat | |
| aatttttttactgattgtttcagaacacccatagttt | |
| ttctctattaaacgttcatccttgacttccccgagtt | |
| tgctggctgaataggaaatttgaagacaaaaaggaaa | |
| gaatcggctcaaacgtcatgcaactgataaggcgacc | |
| gtacttcatttcaaaagaagctcacttactgagcgca | |
| aacattgagaaatgagagcaaaagaagtggttttaca | |
| acatgctaatgtttttagaccttgtacccaataatat | |
| tactgtagtatacagttcggagagcatatggttgaaa | |
| tcttgaaataccaatttatcactagtttgattgtgtt | |
| atcgatgtataaagatatattttatcatttttgatta | |
| ttatctgatattgtggggtgtgaagtaatattatgtg | |
| cgtgtgtggctgattatcgaaaaaaactgaaaattat | |
| caatttttctacaggttatctttttttgttttatttt | |
| tcattattgtattcttcatactccttatcctgcccga | |
| atcacaaaagttccagacaacttgaaggtcgcatcac | |
| gttttgtttataagaatgttgaactggtcagctttgg | |
| agacagaactgtcctccaaatgttgaatatgtttccg | |
| taaaacacactgtgtaacaaaaagcgtaccctttgat | |
| ccggtattcttgaaaaaaaacatagaagggcgtactt | |
| ttcatatctcacaaatatgcgttttttattttataat | |
| aatatcatttattgataattatatgtatttgtatgtt | |
| ttcttgtctttaaaattatggtgtgacatcacagttt | |
| aaaaattatatgaaaactaatgagtgaaaagttaaaa | |
| atgtatagtttgccgtattttctccattagtattgta | |
| ttcaaaacttatttccaattggtgagcactataaact | |
| ttgtagggtaattgaggttcaatactaatttttgaat | |
| atctagcttttgacaaattattcaagttttgacaatg | |
| atgccttatcaaattaaaaaaaattaccataaattct | |
| tgaaagcttttgattttgtttcactctgtgaaggata | |
| gtaatttatcaacattttaactagatacatctgttct | |
| ggcaataaattaaagcaagactattactatgtgcagt | |
| attaatagctgcattgcaacttgtttgaaagtttcag | |
| tgaaaacgcaaaaactcaatttaattttcaagtttgt | |
| ttgcaaaattggatatatgtatccatcaaaagtctga | |
| tcgcctgttttatctacttattgtctcctcagttgat | |
| ttttttgtagttattataaacgctactttgaaaccaa | |
| tattttcttcctcattccacttttttcaattaccaac | |
| taccgtacatgcaatgatgggcggacccgagtcccat | |
| gtttggctggaagtgggtggttgtctgcgtatatgag | |
| cgacagaggtcggggctgaaactgataagaatagtcg | |
| acactaacgccataatcgctagccagccatcatgcac | |
| accgagctcggtgtgcacaccatctttcttttcaaac | |
| caatacgctttgtgccttcattgacaattttcttttt | |
| gataaaatcagcctatctatacttcccaatcattttt | |
| agtcttatcgttgaacagctatcgaggtgccactgtt | |
| ttcactgatatcttctaagttactatggcattaacat | |
| ctttgtgttcttattttatgggttattttaattaatt | |
| tttgcagttaatttttggaatgagcaagaaaatgtta | |
| attgtaatatcttcgtctgaaaattgtcttcaaatag | |
| ttttaattttaaaggcagtatttaagaaaatacactt | |
| ctcgaagcatttttgaatttttgaatttttaaactgc | |
| ttgatgttttaggtgccactgttttcattgataagtt | |
| ttgatgtataaatgcttgattttcttggcattctaat | |
| aaaatagaaactagaaaatagattatagaatggataa | |
| taactacaatgataatgtcaacggctgggccgaaatg | |
| gaaccatctcaaccaatgggaggtctgcgcctaccaa | |
| ctcagaacatggatccaccagagcaaaataatgagtc | |
| acaattgagtgaactaccgagaagtaaacaaaacact | |
| gaaatgcttgtggattataaaattataaaatttcagt | |
| gaaaattgataatgattacgcatctccaattgaacgg | |
| caaagtgttatcacaagtggcacaaataactatgagc | |
| cgaaagtggaaactgttacatcatgtaagtgtgaatt | |
| caag | |

The elt-2 promoter sequence is also available under Genbank Accession No. Z49867.

In an embodiment of the invention, the ges-1 promoter comprises the following nucleotide sequence:

(SEQ ID NO:10)

aagcttaatgaagtttatttcagatcagtaattcga
aatgtttctactggaatccgccaaattgtcgacaac
tcttttaagaaaacgactaaaatgaacttgagtgtt
agcggcgtcttcaccaatacctttagtgacgatgtc
gaagttgactataactgggacaatatcatcgaatat
tcttacaaatcttcggaaacggagtataagaaaaga
gaggacgatggagaccatacggaaatagctgttaga
gagctcggaattcagctcaacaatccgaatcatcag
ctggagtacctggaattctggataaaacctttacga
tattttaccgaagaagaaaggaccgaacgatggaat
tgtctggaaaaattcttcaagaaggttgagaactcg
aaggttgctaaaatctcgttcgttggattcagggga
tataaaattgcaacgattttgcaatctttctcacca
ggaaccctgacacgactttatgtcaatgttgctgat
tcggatatctctcaaatcatcgacgcggagcattgg
atttatctaaaaaagatcagagtgctttcgccttct
ccggtttccattccgattgatgctattcttcacatg
acacacttcgacgtttctgtgatccacctttccact
gatgatgtcgcaaagatcaaaaatgtgagtgaacaa
taaaaagttgaaaaaacattattttaaaataattac
tggtcgttctcgaatccgcaagtttcacaatgggga
tgatccgtaccaacatcacggacccaatggaaactg
caaaaatcttcggaacgcacattcatcgtgatgatg
tggatgttttgggtgacatggatattaacaaggacg
atggtccagaagttcatttggatgacgattggcatg
aggaggagctcgtgggggctgtgggggcggaggagg
aggaggacgaggaggacgaggaagaggaggacgagg
acgaggaagaggggatggctgatgtcccggaagtca
atgaggccgtgcacagagtcaacattaaactcggcg
acgctgacatcaccgttgtggtgtctccgacatact
tgaagattgcaaagaatactcgtgaaaattgaactt
taattcctttctctttttttttgactcaaaaaataa
atagtatttcaaaggaatcgcatttcaaactgatac
ttaagtttttcaataggttttttttttcagttttga
tttttttttgtgcttaaagagaagactgttagccac
tcagccacttcagccatatcgaaacagtcatattgg
ctctctttcagatcacttaaaactccgaactatgat
gacgaaaaaatgttttcttgaattttcattttattg
tgaaattgaaaactctgcatagcccgcagtcacttg
aaagcacatttaagatggaatcaaatggtaagaaag
ctcgtgtaatcaggagattttatccggggtatgccg
aggaatatatggtgcactcgcattagctggcatttt
ttgtaattatcggatttctacctttttcttaatcta
gtttgttatcattgtccatcatttacaaccaaaaat
ttgcgtcatttacaaccaaaaattgatctgacaatt
atcataattcggtttttattttcagaagaaacgctc
cgatgtgatcctatagtgaatatacttttccatcct
ctctgaaataaacaattatgaagaatgcatcaattt
gcattcgaaataactgccgtattttgtctattggta
tggctgcagtaccatttttcttcgcgtctttgcgca
gccttcgggcgctaccaataaggctaagccggggta
agttatttcagaattc

The ges-1 promoter sequence is also available under Genbank Accession No. AF039046.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

A “mutation” or “double mutation” refers to a change in the genetic material of an organism (e.g., C.elegans worm) that results in expression of a phenotype not observed in the parental organism. In an embodiment of the invention, a ncr-2, ncr-1 double mutation refers to mutations that result in a decrease, to any degree, of the function, activity or expression of ncr-2 and ncr-1, for example, an increase, to any degree, in the likelihood that the worm will express the dauer phenotype as compared to that of the parental worm. A mutation of a particular gene includes full knock-outs of the gene, its promoter or other associated regulatory elements, as well as point mutations, internal deletions, truncations, interruptions (e.g., insertion of a heterologous sequence) and frame-shifts thereof.

The meaning of the term “functional NPC1L1” would be clear to any practitioner of ordinary skill in the art. For example, in an embodiment, the term refers to NPC1L1 polypeptide (e.g., human NPC1L1 such as SEQ ID NO: 6) that comprises any detectable level of activity such as binding to cholesterol, ezetimibe or any derivative of ezetimibe (e.g., SCH354909 (see e.g., Altmann et al., Biochim Biophys Acta. 1580(1):77-93 (2002)), in vivo intestinal cholesterol or sitosterol absorption or macrophage cholesterol uptake (see e.g., Altmann et al., Science 303(5661):1201-1204 (2004); Davis et al., J. Biol. Chem. 279(32): 33586-33592 (2004) or Seedorf et al., Biochem Biophys Res Commun. 320(4):1337-1341 (2004)). Other assays for confirming NPC1I1 functionality are set forth in published U.S. patent application no. 2004/0161838.

A “polynucleotide”, “nucleic acid” or “nucleic acid molecule” includes the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in single stranded form, double-stranded form or otherwise.

A “polynucleotide sequence”, “nucleic acid sequence” or “nucleotide sequence” is a series of nucleotide bases (also called “nucleotides”) in a nucleic acid, such as DNA or RNA, and means any chain of two or more nucleotides.

A “coding sequence” or a sequence “encoding” an expression product, such as a RNA, polypeptide, protein, or enzyme, is a nucleotide sequence that, when expressed, results in production of the product.

The term “gene” means a DNA sequence that codes for or corresponds to a particular sequence of ribonucleotides or amino acids which comprise all or part of one or more RNA molecules, proteins or enzymes, and may or may not include regulatory DNA sequences, such as promoter sequences, which determine, for example, the conditions under which the gene is expressed. Genes may be transcribed from DNA to RNA which may or may not be translated into an amino acid sequence.

As used herein, the term “oligonucleotide” refers to a nucleic acid, generally of no more than about 100 nucleotides (e.g., 30, 40, 50, 60, 70, 80, or 90), that may be hybridizable to a genomic DNA molecule, a cDNA molecule, or an mRNA molecule encoding a gene, mRNA, cDNA, or other nucleic acid of interest. Oligonucleotides can be labeled, e.g., by incorporation of ³²P-nucleotides, ³H-nucleotides, ¹⁴C-nucleotides, ³⁵S-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of the gene, or to detect the presence of nucleic acids. Generally, oligonucleotides are prepared synthetically, preferably on a nucleic acid synthesizer.

A “protein sequence”, “peptide sequence” or “polypeptide sequence” or “amino acid sequence” may refer to a series of two or more amino acids in a protein, peptide or polypeptide. “Protein”, “peptide” or “polypeptide” includes a contiguous string of two or more amino acids.

The terms “isolated polynucleotide” or “isolated polypeptide” include a polynucleotide (e.g., RNA or DNA molecule, or a mixed polymer) or a polypeptide, respectively, which are partially or fully separated from other components that are normally found in cells or in recombinant DNA expression systems. These components include, but are not limited to, cell membranes, cell walls, ribosomes, polymerases, serum components and extraneous genomic sequences.

An isolated polynucleotide or polypeptide will, preferably, be an essentially homogeneous composition of molecules but may contain some heterogeneity.

“Amplification” of DNA as used herein may denote the use of polymerase chain reaction (PCR) to increase the concentration of a particular DNA sequence within a mixture of DNA sequences. For a description of PCR see Saiki, et al., Science (1988) 239:487.

The term “host cell” includes any cell of any organism that is selected, modified, transfected, transformed, grown, or used or manipulated in any way, for the production of a substance by the cell, for example the expression or replication, by the cell, of a gene, a DNA or RNA sequence or a protein. Preferred host cells include C.elegans cells.

The nucleotide sequence of a nucleic acid may be determined by any method known in the art (e.g., chemical sequencing or enzymatic sequencing). “Chemical sequencing” of DNA includes methods such as that of Maxam and Gilbert (1977) (Proc. Natl. Acad. Sci. USA 74:560), in which DNA is randomly cleaved using individual base-specific reactions. “Enzymatic sequencing” of DNA includes methods such as that of Sanger (Sanger, et al., (1977) Proc. Natl. Acad. Sci. USA 74:5463).

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

In general, a “promoter” or “promoter sequence” is a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a nucleic acid of the invention. Promoters which may be used to control gene expression include, but are not limited to, the C.elegans ncr-1 promoter or the ncr-2 promoter or any adult-specific C.elegans promoter including, but by no means limited to, the col-19 promoter (gene also called ZK1193.1) (Abrahante et al. Genetics 149:1335-1351 (1998); Thein et al. Developmental Dynamics 226:5239-5253 (2003)) and the vit-2 promoter (gene also called C42D8.2) (Grant, B. and Hirsh, D. Molecular Biology of the Cell 10:4311-4326 (1999)). Other promoters include the cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the β-lactamase promoter (Villa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal 4 promoter, the ADC (alcohol dehydrogenase) promoter, PGK (phosphoglycerol kinase) promoter or the alkaline phosphatase promoter.

A coding sequence is “under the control of”, “functionally associated with” or “operably associated with” or “linked” to transcriptional and translational control sequences in a cell when the sequences direct RNA polymerase mediated transcription of the coding sequence into RNA, preferably mRNA, which then may be RNA spliced (if it contains introns) and, optionally, translated into a protein encoded by the coding sequence.

The terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein. The expression product itself may also be said to be “expressed” by the cell.

The term “transformation” means the introduction of a nucleic acid into a cell. The introduced gene or sequence may be called a “clone”. A host cell that receives the introduced DNA or RNA has been “transformed” and is a “transformant” or a “clone.” The DNA or RNA introduced to a host cell can come from any source, including cells of the same genus or species as the host cell, or from cells of a different genus or species. C.elegans worms can be transformed with DNA from any number of techniques that are known in the art including microinjection and microparticle bombardment (discussed infra).

The term “vector” includes a vehicle (e.g., a plasmid) by which a DNA or RNA sequence can be introduced into a host cell, so as to transform the host and, optionally, promote expression and/or replication of the introduced sequence.

Vectors that can be used in this invention include plasmids, viruses, bacteriophage, integratable DNA fragments, and other vehicles that may facilitate introduction of the nucleic acids into the genome of the host. Plasmids are the most commonly used form of vector but all other forms of vectors which serve a similar function and which are, or become, known in the art are suitable for use herein. See, e.g., Pouwels, et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass.

Several C.elegans vectors useful for maintaining pieces of DNA in a C.elegans cell are known in the art. For example, BD Biosciences (San Jose, Calif.) sells the following C.elegans vectors: pTU#60-GFP, pTU#61-GFP, pTU#62-GFP and pTU#63-GFP. Other C.elegans vectors are disclosed by Fire et al. (Nuc. Acid. Res. 18(14): 4269-4270 (1990)): pAST18a, pAST18b, pAST19b, pAST19a, pPD26.14, pICT19h, pICT19r and pAST (see EMBL Accession Nos X53121-X53127). In an embodiment of the invention, DNA can be introduced into a C.elegans cell using the pPD49.26 vector (Fire et al. Gene 93(2): 189-198 (1990)).

The term “expression system” means a host cell and compatible vector which, under suitable conditions, can express a protein or nucleic acid which is carried by the vector and introduced to the host cell. Common expression systems include E. coli host cells and plasmid vectors, insect host cells and Baculovirus vectors, and mammalian host cells and vectors.

The present invention contemplates any superficial or slight modification to the amino acid or nucleotide sequences which correspond to the polypeptides of the invention. For example, the sequences of ncr-1, ncr-2 and NPC1L1, polypeptides and polynucleotide that can be used in the present invention, are set forth above (SEQ ID NOs: 1-6); the present invention contemplates any embodiment (e.g., a functional assay or a transgenic C.elegans worm) comprising ncr-1, ncr-2 and/or NPC1L1 sequences set forth herein as well as embodiments comprising any superficial or slight modification of these sequences. In particular, the present invention contemplates sequence conservative variants of the nucleic acids which encode the polypeptides of the invention. “Sequence-conservative variants” of a polynucleotide sequence are those in which a change of one or more nucleotides in a given codon results in no alteration in the amino acid encoded at that position. Function-conservative variants of the polypeptides of the invention are also contemplated by the present invention. “Function-conservative variants” are those in which one or more amino acid residues in a protein or enzyme have been changed without altering the overall conformation and function of the polypeptide, including, but, by no means, limited to, replacement of an amino acid with one having similar properties. Amino acids with similar properties are well known in the art. For example, polar/hydrophilic amino acids which may be interchangeable include asparagine, glutamine, serine, cysteine, threonine, lysine, arginine, histidine, aspartic acid and glutamic acid; nonpolar/hydrophobic amino acids which may be interchangeable include glycine, alanine, valine, leucine, isoleucine, proline, tyrosine, phenylalanine, tryptophan and methionine; acidic amino acids which may be interchangeable include aspartic acid and glutamic acid and basic amino acids which may be interchangeable include histidine, lysine and arginine.

The present invention includes embodiments (e.g., functional assays or transgenic C.elegans worms) comprising polynucleotides encoding C.elegans ncr-1, C.elegans ncr-2 and rat, human or mouse NPC1L1 and fragments thereof as well as nucleic acids which hybridize to the polynucleotides. Preferably, the nucleic acids hybridize under low stringency conditions, more preferably under moderate stringency conditions and most preferably under high stringency conditions. A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook, et al., supra). The conditions of temperature and ionic strength determine the “stringency” of the hybridization. Typical low stringency hybridization conditions are 55° C., 5×SSC, 0.1% SDS, 0.25% milk, and no formamide at 42° C.; or 30% formamide, 5×SSC, 0.5% SDS at 42° C. Typical, moderate stringency hybridization conditions are similar to the low stringency conditions except the hybridization is carried out in 40% formamide, with 5× or 6×SSC at 42° C. High stringency hybridization conditions are similar to low stringency conditions except the hybridization conditions are carried out in 50% formamide, 5× or 6×SSC and, optionally, at a higher temperature (e.g., higher than 42° C.: 57° C., 59° C., 60° C., 62° C., 63° C., 65° C. or 68° C.). In general, SSC is 0.15M NaC1 and 0.015M Na-citrate. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook, et al., supra, 9.50-9.51). For hybridization with shorter nucleic acids, i.e., oligonucleotides, the position of mismatches becomes more important, and the length of the oligonucleotide determines its specificity (see Sambrook, et al., supra).

Also included in the present invention are embodiments (e.g., functional assays or transgenic C.elegans worms) comprising nucleotide sequences and polypeptides comprising amino acid sequences which are at least about 70% identical, preferably at least about 80% identical, more preferably at least about 90% identical and most preferably at least about 95% identical (e.g., 95%, 96%, 97%, 98%, 99%, 100%) to the reference ncr-1, ncr-2 or NPC1L1 nucleotide (e.g., SEQ ID NOs: 1, 3 or 5) and ncr-1, ncr-2, NPC1L1 amino acid sequences (e.g., SEQ ID NO: 2, 4 or 6), when the comparison is performed by a BLAST algorithm wherein the parameters of the algorithm are selected to give the largest match between the respective sequences over the entire length of the respective reference sequences. Polypeptides comprising amino acid sequences which are at least about 70% similar, preferably at least about 80% similar, more preferably at least about 90% similar and most preferably at least about 95% similar (e.g., 95%, 96%, 97%, 98%, 99%, 100%) to the reference ncr-1, ncr-2 or NPC1L1 amino acid sequence of SEQ ID NO: 2, 4 or 6, when the comparison is performed with a BLAST algorithm wherein the parameters of the algorithm are selected to give the largest match between the respective sequences over the entire length of the respective reference sequences, are also included in the present invention.

Sequence identity refers to exact matches between the nucleotides or amino acids of two sequences which are being compared. Sequence similarity refers to both exact matches between the amino acids of two polypeptides which are being compared in addition to matches between nonidentical, biochemically related amino acids. Biochemically related amino acids which share similar properties and may be interchangeable are discussed above.

The following references regarding the BLAST algorithm are herein incorporated by reference: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M., et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp.1-14, Plenum, N.Y.

### C.elegans Worms

The scope of the present invention includes an ncr-2, ncr-1 double mutant C.elegans worm having functional NPC1L1 polypeptide (e.g., human NPC1L1, for example SEQ ID NO: 6). The NPC1L1 in the double mutant worms can be from any organism including rat, mouse and human. In an embodiment of the invention, the ncr-2, ncr-1 double mutant C.elegans worm has human NPC1L1 (e.g., SEQ ID NO: 6). Embodiments of the invention include ncr-2, ncr-1 double mutant C.elegans worms having the NPC1L1 gene (e.g., human NPC1L1 for example, SEQ ID NO: 5) e.g., in a plasmid vector or integrated into a C.elegans chromosome, operably linked to a promoter, for example, a C.elegans ncr-1 promoter or ncr-2 promoter or a gut-specific promoter such as e/t-2 promoter (Fukushige et al. Dev. Biology 198: 286-302 (1998)) or ges-1 promoter (Kennedy et al. J. Mol. Bio. 229(4): 890-908(1993)). In an embodiment of the invention, a C.elegans worm of the invention has the genetic background of strain N2 (Brenner Genetics 77: 71-94 (1974)), an ncr-2, ncr-1 double mutation and functional NPC1L1 (e.g., human NPC1L1 such as SEQ ID NO: 6). In another embodiment of the invention, a C.elegans worm of the invention is the ncr-2, ncr-1 double mutant described by Sym et al. (Current Biology 10:527-530 (2000)) comprising functional NPC1L1 (e.g., human NPC1L1 for example, SEQ ID NO: 6).

The scope of the present invention also includes any C.elegans worm, for example a wild-type C.elegans worm (e.g., strain N2), comprising NPC1L1 (e.g., human NPC1L1 for example, SEQ ID NO: 6). Such worms are useful, for example, for the recombinant production and isolation of NPC1L1 (e.g., human NPC1L1).

The present invention also includes any of the C.elegans worms described herein, for example, Strain 2a, Strain 2b, Strain 3, Strain 4a or Strain 4b (see infra).

The scope of the present invention includes any C.elegans worm described herein (e.g., ncr-2, ncr-1 double mutant comprising human NPC1L1) as well as any product isolated from such a worm including, but not limited to, individual cells taken from the worm. Moreover, the scope of the present invention includes any C.elegans worm described herein in any growth stage including egg, L1, L2, L2d, L3, L4, dauer and adult.

In an embodiment of the invention, NPC1L1, in a worm of the invention, is operably linked to, for example, any C.elegans promoter, such as an adult specific promoter including, but not limited to, the col-19 promoter (gene also called ZK1193.1) (Abrahante etal. Genetics 149:1335-1351 (1998); Thein etal Developmental Dynamics 226:5239-5253 (2003)) or the vit-2 promoter (gene also called C42D8.2) (Grant, B. and Hirsh, D. Molecular Biology of the Cell 10:4311-4326 (1999)).

Growth and propagation of C.elegans worms can be performed using standard techniques that are well known in the art. For example, standard culture and handling techniques for C.elegans are discussed in Sulston & Hodgkin (Methods. In The Nematode Caenorhabditis elegans; Ed. Wood W B. Cold Spring Harbor: Cold Spring Harbor Laboratory Press; 1988) and Brenner, Genetics 77: 71-94 (1974).

For example, a C.elegans worm can be maintained on an NGM (nematode growth medium) agar plate comprising 51.3 mM NaCl, 0.25% Bactopeptone, 1.7% Bacto-agar, 0.0005% cholesterol, 1 mM CaCl₂, 1 mM MgSO₄ and 25 mM potassium phosphate, pH 6.0) seeded with E.coli cells (see e.g., Brenner Genetics 77: 71-94 (1974)). Other media include (i) 3% Yeast extract, 3% soy peptone and 10% heated liver extract; (ii) 3% Gistex yeast extract, 3% soy peptone and 10% heated liver extract; (iii) 3% Gistex yeast extract, 3% soy peptone, 1% dextrose, 1% bacto-casitone and 500 μg/ml haemoglobin; (iv) 3% Gistex yeast extract, 3% soy peptone, 1% dextrose, 50 μg/ml acid precipitated haemin chloride; (v) 6% Gistex yeast extract, 1% dextrose, 1% bacto-casitone, 50 μg/ml acid precipitated haemin chloride; (vi) 6% Gistex yeast extract, 1% dextrose, 50 μg/ml acid precipitated haemin chloride; and (vii) 5% Gistex yeast extract, 1% dextrose, 50 μg/ml acid precipitated haemin chloride (see e.g., Vanfleteren, Experientia 32(8): 1087-1088 (1976)).

There are several protocols known in the art for transformation of C.elegans with nucleic acids. For example, a microinjection method is described by Stinchcomb et al., Mol. Cell. Bio. 5(12): 3484-3496 (1985), Mello et al. (EMBO J. 10(12):3959-70 (1991)) and by Kimble et al., Nature (London) 299: 456-458 (1982). Jefferson et al. describe a method for expressing chimeric genes in C.elegans (J. Mol. Biol. 193(1):41-46 (1987)). Hull et al., describe a method for injection of double stranded RNA into C.elegans (Methods Mol. Biol. 265:23-58 (2004)). Wilm et al. (Gene 229(1-2):31-35 (1999)) describe ballistic transformation (microparticle bombardment) of C.elegans worms. Moreover, Praitis et al. (Genetics 157:1217-1226 (2001)) disclose methods for generation of chromosomal integration mutants of C.elegans. Alternatively, a plasmid vector (see supra) can be used to maintain a polynucleotide in a C.elegans cell episomally.

A convenient method by which a C.elegans transformant can be identified is by using the dominant rol-6 allele that causes a readily distinguished roller phenotype in transgenic animals, as a co-transformation marker (Kramer et al., Mol. Cell. Biol. 10: 2081-2089 (1990)). For example, the plasmid pRF4 contains the rol-6 gene (Mello et al. EMBO J. 10(12): 3959-3970 (1991)).

### Assays

The present invention includes cellular assay methods by which inhibitors of NPC1L1 can be identified rapidly and conveniently. Without limiting the present invention to a single theory or mechanism of action, in general, the assays are based on the finding that NPC1L1 (e.g., SEQ ID NO: 6) complements the ncr-1, ncr-2 double mutant in C.elegans worms. Ncr-2, Ncr-1 double mutant worms exhibit the dauer phenotype whereas; however, expression of a NPC1L1 (e.g., SEQ ID NO: 6) in the double mutant results in rescue of the worms from the dauer phenotype and expression of an adult phenotype. Inhibition of NPC1L1, for example, by contacting the protein with an inhibitory substance (e.g., ezetimibe), results in reversal of the NPC1L1-dependent rescue from the dauer phenotype.

For example, in an embodiment of the invention, the presence of an NPC1L1 inhibitor is indicated by the expression of a dauer phenotype by the C.elegans worm being assayed. Dauer C.elegans larva can be easily identified, visually, under a microscope (e.g., a dissecting microscope), by any practitioner of ordinary skill in the art. C.elegans dauer larva is a developmentally arrested dispersal stage that may be formed under conditions of starvation or overcrowding. In general, dauer worms have a longer, thinner body shape than L2 larva and are less active. Typically, dauer larva have a closed mouth and do not feed. An example of an assay method for identifying a substance that inhibits cholesterol absorption (e.g., intestinal absorption), reduces elevated total cholesterol, reduces elevated low density lipoprotein cholesterol, reduces elevated apolipoprotein B, treats or prevents heterozygous familial hypercholesterolemia, treats or prevents non-familial hypercholesterolemia, treats or prevents homozygous familial hypercholesterolemia, treats or prevents homozygous sitosterolemia or that inhibits NPC1L1 (e.g., human NPC1L1, e.g., SEQ ID NO: 6) comprises the steps of: (a) contacting a C.elegans worm lacking functional ncr-1 (e.g., SEQ ID NO: 2) and ncr-2 polypeptide (e.g., SEQ ID NO: 4) and having functional NPC1L1 (e.g., SEQ ID NO: 6) with the substance to be tested; and (b) determining if the worm exhibits a dauer phenotype (e.g., as identified visually under a microscope); whereby the substance is selected if the dauer phenotype is observed. For example, the larva can be observed once or several times over the course of several days (e.g., 1, 2, 3 or 4 days) following exposure of the worms to the substance being tested for detection of the dauer phenotype. In an embodiment, worms are observed once 3 days after exposure of the worms to the substance being tested. In an embodiment of the invention, an optional negative-control assay is performed in conjunction with this assay and comprises the steps of: (a) contacting a C.elegans worm lacking functional ncr-1 (e.g., SEQ ID NO: 2) and ncr-2 polypeptide (e.g., SEQ ID NO: 4) and having functional NPC1L1 polypeptide (e.g., SEQ ID NO: 6) with a blank substance known to not inhibit cholesterol absorption (e.g., intestinal absorption) or not to inhibit NPC1L1 (e.g., human NPC1L1, e.g., SEQ ID NO: 6); and (b) determining if the worm exhibits a dauer phenotype (e.g., as identified visually under a microscope). Confirmation that the assay is functioning properly is provided if the worm is confirmed not to exhibit the dauer phenotype in the negative-control experiment in the presence of the blank substance. In an embodiment of the invention, an optional, positive-control assay is performed in conjunction with the assay and comprises the steps of (a) contacting a C.elegans worm lacking functional ncr-1 (e.g., SEQ ID NO: 2) and ncr-2 polypeptide (e.g., SEQ ID NO: 4) and having functional NPC1L1 polypeptide (e.g., SEQ ID NO: 6) with a positive-control substance known to inhibit cholesterol absorption (e.g., intestinal absorption) or to inhibit NPC1L1 (e.g., human NPC1L1, e.g., SEQ ID NO: 6); for example, ezetimibe; and (b) determining if the worm exhibits a dauer phenotype (e.g., as identified visually under a microscope). Confirmation that the assay is functioning properly is provided if the worm is confirmed to exhibit the dauer phenotype in the positive-control experiment in the presence of the positive-control substance.

Another method by which C.elegans worms can be identified as adult, non-dauer larva is by determining the presence of a chitinase. Chitinase production is a marker for adult, non-dauer worms (see e.g., Wu et al., J. Biol. Chem. 276 (45): 42557-42564 (2001)). An example of an assay method for identifying a substance that inhibits cholesterol absorption (e.g., intestinal absorption), that inhibits NPC1L1 (e.g., human NPC1L1, e.g., SEQ ID NO: 6), reduces elevated total cholesterol, reduces elevated low density lipoprotein cholesterol, reduces elevated apolipoprotein B, treats or prevents heterozygous familial hypercholesterolemia, treats or prevents non-familial hypercholesterolemia, treats or prevents homozygous familial hypercholesterolemia, or treats or prevents homozygous sitosterolemia comprises the steps of: (a) contacting a C.elegans worm lacking functional ncr-1 (e.g., SEQ ID NO: 2) and ncr-2 polypeptide (e.g., SEQ ID NO: 4) and having functional NPC1L1 (e.g., SEQ ID NO: 6) with the substance to be tested; and (b) determining whether the worm secretes chitinase; whereby the substance is selected if chitinase is not secreted. In an embodiment, chitinase production can be measured once or several times over a period of time (e.g., 1, 2, 3 or 4 days) following exposure of the worms to the substance being tested. In an embodiment, chitinase expression is measured once 3 days after exposure of the worms to the substance being tested. In an embodiment of the invention, an optional negative-control experiment is performed in conjunction with the assay comprising the following steps: (a) contacting a C.elegans worm lacking functional ncr-1 (e.g., SEQ ID NO: 2) and ncr-2 polypeptide (e.g., SEQ ID NO: 4) and having functional NPC1L1 (e.g., SEQ ID NO: 6) with a blank substance known not to inhibit cholesterol absorption (e.g., intestinal absorption) or not to inhibit NPC1L1 (e.g., human NPC1L1, e.g., SEQ ID NO: 6); and (b) determining whether the worm secretes chitinase. Confirmation that the assay is functioning properly is provided if the worm is confirmed to secrete chitinase in the negative-control experiment in the presence of the blank substance. In an embodiment of the invention, an optional positive-control experiment is performed in conjunction with the assay comprising the following steps: (a) contacting a C.elegans worm lacking functional ncr-1 (e.g., SEQ ID NO: 2) and ncr-2 polypeptide (e.g., SEQ ID NO: 4) and having functional NPC1L1 (e.g., SEQ ID NO: 6) with a substance known to inhibit cholesterol absorption (e.g., intestinal absorption) or to inhibit NPC1L1 (e.g., human NPC1L1, e.g., SEQ ID NO: 6); for example ezetimibe and (b) determining whether the worm secretes chitinase. Confirmation that the assay is functioning properly is provided if the worm is confirmed not to secrete chitinase in the positive-control experiment in the presence of the positive-control substance.

Another method for determining whether the dauer phenotype is expressed in a C.elegans worm being assayed is to determine whether one or more adult-specific promoters are being expressed. If the adult-specific promoter is expressed, this indicates that the worm is an adult and not dauer larva. If the adult-specific promoter is not expressed, this indicates that the worm is not adult and is, instead, a dauer larva. Expression from the adult-specific promoter can be identified by operably linking the promoter to a reporter gene and determining whether the reporter gene is expressed. An example of an assay method for identifying a substance that inhibits cholesterol absorption (e.g., intestinal absorption), inhibits NPC1L1 (e.g., human NPC1L1, e.g., SEQ ID NO: 6), reduces elevated total cholesterol, reduces elevated low density lipoprotein cholesterol, reduces elevated apolipoprotein B, treats or prevents heterozygous familial hypercholesterolemia, treats or prevents non-familial hypercholesterolemia, treats or prevents homozygous familial hypercholesterolemia, or treats or prevents homozygous sitosterolemia comprises the steps of: (a) contacting a C.elegans cell lacking functional ncr-1 (e.g., SEQ ID NO: 2) and ncr-2 polypeptide (e.g., SEQ ID NO: 4), having functional NPC1L1 polypeptide (e.g., SEQ ID NO: 6) and having an adult-specific C.elegans promoter operably linked to a reporter with the substance to be tested; and (b) determining whether the reporter is expressed; whereby the substance is selected if the reporter is not expressed. The reporter to which the adult-specific promoter is linked can be any suitable reporter known in the art including, but by no means limited to, any of those discussed herein. In an embodiment, reporter expression can be measured once or several times over a period of time (e.g., 1, 2, 3 or 4 days) following exposure of the worms to the substance being tested. In an embodiment, reporter expression is measured once 3 days after exposure of the worms to the substance being tested. In an embodiment of the invention, an optional negative-control experiment is performed in conjunction with the assay comprising the following steps: (a) contacting a C.elegans cell lacking functional ncr-1 (e.g., SEQ ID NO: 2) and ncr-2 polypeptide (e.g., SEQ ID NO: 4), having functional NPC1L1 polypeptide (e.g., SEQ ID NO: 6) and having an adult-specific C.elegans promoter operably linked to a reporter with the a blank substance known not to inhibit cholesterol absorption (e.g., intestinal absorption) or to inhibit NPC1L1 (e.g., human NPC1L1, e.g., SEQ ID NO: 6); and (b) determining whether the reporter is expressed. Confirmation that the assay is functioning properly is provided if expression by the promoter is detected in the negative-control experiment in the presence of the blank substance. In an embodiment of the invention, an optional positive-control experiment is performed in conjunction with the assay comprising the following steps: (a) contacting a C.elegans cell lacking functional ncr-1 (e.g., SEQ ID NO: 2) and ncr-2 polypeptide (e.g., SEQ ID NO: 4), having functional NPC1L1 polypeptide (e.g., SEQ ID NO: 6) and having an adult-specific C.elegans promoter operably linked to a reporter with a substance known to inhibit cholesterol absorption (e.g., intestinal absorption) or that inhibits NPC1L1 (e.g., human NPC1L1, e.g., SEQ ID NO: 6); for example ezetimibe and (b) determining whether the reporter is expressed. Confirmation that the assay is functioning properly is provided if expression by the promoter is not detected in the positive-control experiment.

In another embodiment of the invention, an optional negative-control assay is performed in conjunction with any of the assays described herein and comprises the steps of: (a) contacting a wild-type C.elegans worm comprising functional ncr-1 (e.g., SEQ ID NO: 2) and ncr-2 polypeptide (e.g., SEQ ID NO: 4) (e.g., C.elegans strain N2) with the substance being tested for the ability to inhibit cholesterol absorption (e.g., intestinal absorption) or to inhibit NPC1L1 (e.g., human NPC1L1, e.g., SEQ ID NO: 6); and (b) determining if the worm exhibits a dauer phenotype (e.g., as identified visually under a microscope). Exhibition of a dauer phenotype by a wild-type worm contracted with the substance being tested indicates that the substance induces the dauer phenotype and may not necessarily inhibit NPC1L1.

A reporter to which an adult-specific promoter used in an assay described herein includes any gene or protein that allows detection of expression from the promoter. A non-limiting list of reporter genes that may be operably associated with an adult-specific promoter as discussed herein includes, but is not limited to, red bioluminescent proteins from Anthozoa, Photorhabdus luminescens LuxA-E, FMN oxidoredtuctase, Pyrophorus plagiophthalamus luciferase, Chloramphenicol Acetyltransferase (CAT), β-Galactosidase (β-Gal), Vibrio harveyi luciferase, Photinus pyralis Luciferase, Renilla reniformis luciferase, Green Fluorescent Protein (GFP; and mutant variations thereof), β-glucuronidase (GUS), chitinase and epitope tags. Typically, the expression of a reporter gene (e.g., by an adult-specific C.elegans promoter) is detected by detecting the enzymatic activity of the reporter (e.g., by detecting luminescence of a luciferase reporter). The term reporter also includes, for example, any gene or open reading frame that can be detected when it is expressed; for example, by detecting the mRNA by northern blot analysis or by detecting the translated protein by western blot analysis. For example, the term “reporter” includes any gene that is naturally located downstream of or is controlled by a C.elegans promoter (e.g., within the wild-type N2 strain's genome) to which it is operably associated. For example, in an embodiment of the invention, a reporter linked to the Col-19 promoter is the Col-19 gene (ZK1193.1) itself. Detection of any of the foregoing reporters can be performed by any of many methods that are well known in the art.

A Photorhabdus luminescens LuxA-E, FMN oxidoredtuctase construct such as that disclosed in published U.S. Patent Application no. US20040142356 can be operably associated with a C.elegans promoter (e.g., adult specific promoter). P.luminescens luciferase luminescence can be detected by detecting light emission at 490 nm.

Various bioluminescent proteins from Anthozoa can also be operably associated with a C.elegans promoter (e.g., adult-specific promoter). For example, Matz et al. (Nat. Biotech. 17:969-973 (1999)) discloses bioluminescent proteins including amFP486 from Anemonia majano (Genbank Accession No. AF168421), zFP506 (Genbank Accession No. AF168422) and zFP538 (Genbank Accession No. AF168423) from Zoanthus sp., dsFP483 from Discosoma striata (Genbank Accession No. AF168420), drFP583 from Discosoma sp. “red” (Genbank Accession No. AF168419) and cFP484 from Clavularia sp (Genbank Accession No. AF168424) (see also Gross et al. Proc. Natl. Acad. Sci. 97(22): 11990-11995 (2000) and Fradkov et al. FEBS Letters 479: 127-130 (2000)). Methods for detection of the Anthozoa proteins are discussed in Matz et al.; the disclosure of which is incorporated by reference.

Click beetle (Pyrophorus plagiophthalamus) luciferase, such as that disclosed by Wood et al., Science 244(4905):700-702 (1989), can also be operably associated with a C.elegans promoter (e.g., adult specific promoter).

Chloramphenicol Acetyltransferase (CAT) can be operably associated with a C.elegans promoter (e.g., an adult-specific promoter). CAT comes from microorganisms and catalyzes the transfer of acetyl groups from acetyl coenzyme A to chloramphenicol. In general, in a CAT assay, a CAT-containing lysate of a transfected cell is incubated with ¹⁴C-chloramphenicol, which is then acetylated. Acetylated and non-acetylated ¹⁴C-chloramphenicol can then be separated using thin-layer chromatography and visualized by autoradiography. If necessary, the distribution of radioactivity can be quantified by a scanning system. Other methods of carrying out CAT assays are well known in the art.

The prokaryotic β-galactosidase (β-gal) can also be operably associated with a C.elegans promoter (e.g., an adult-specific promoter). β-gal naturally catalyzes the hydrolysis of β-galactosides (e.g., lactose). However, the use of non-physiological substrates also enables the quantification of β-galactosidase activity in lysates of transfected cells via spectrophotometry (e.g., with 0-nitrophenyl-β-D-galactoside=ONPG), fluorometry (e.g., with a 4-methyl-umbelliferyl-β-galactopyranoside compound=MUG) or via chemiluminescence. Detection by chemiluminescence (e.g, with 1.2 dioxetan-galactopyranoside derivatives) is 100-1,000 times more sensitive than the other two detection methods, and thus even more sensitive than the luciferase assay. A major advantage of this system is the fact that β-galactosidase activity can also be measured in situ. Methods for detecting β-gal activity are well known in the art.

V.harveyi luciferase is a dimeric protein comprising an alpha and a beta subunit encoded by luxA and luxB, such as that disclosed by Johnston et al., J. Biol. Chem. 261(11): 4805-4811 (1986), that can be operably associated with a C.elegans promoter (e.g., adult specific promoter). If only one of luxA and luxB are fused to the adult-specific promoter, then the other must be expressed in the cell constitutively. V.harveyi luciferase luminescence can be detected by detecting light at 490 nm.

The luciferase gene from the North American firefly (Photinus pyralis) such as that disclosed by DeWet et al., Mol. Cell. Biol. 7(2): 725-737 (1987) can be operably associated with a C.elegans promoter (e.g., adult-specific promoter). P.pyralis luciferase catalyzes a bioluminescence reaction. In the luciferase assay, the lysates of transfected cells are incubated with luciferin, molecular oxygen, ATP and Mg²⁺. The luciferase catalyzes the oxidation of luciferin in oxyluciferin and CO₂. In this reaction, light with a wavelength of 562 nm is emitted, which then fades rapidly. The light emitted can be measured in a luminometer or in a liquid-scintillation counter. Light emission is proportional to the amount of luciferase in the lysate. The sensitivity of the luciferase assay is further increased by adding co-enzyme A to the reaction preparation, rendering it 10-20 times more sensitive than the CAT assay.

Luciferase from Renilla (Renilla reniformis), such as that disclosed by Lorenz et al., Proc. Natl. Acad. Sci. 88: 4438-4492 (1991), can also be operably associated with a C.elegans promoter (e.g., an adult-specific promoter). The activities of firefly and Renilla luciferase can be measured separately in one sample. The activity of the Renilla luciferase can therefore be used as an internal control for comparing different transfection experiments. Both luciferases are also used in co-transfection experiments for the parallel examination of two cis elements.

Aequorea victoria green fluorescent protein (GFP), such as that disclosed by Prasher et al. (Gene 111 (2):229-33 (1992)), can be operably associated with a C.elegans promoter (e.g., an adult-specific promoter). The green fluorescent protein from the A.victoria jellyfish requires no additional proteins, substrates or co-factors to emit light. When irradiated with UV light or blue light, it emits green light, which enables the examination of gene expression and protein localization in situ and in vivo. In addition, the gene expression can be observed in real time. A.victoria GFP can be detected by exciting fluorescence with 485 nm light and measuring light output at 535 nm.

Variations of GFP with different absorption and emission maxima can also be operably associated with a C.elegans promoter (e.g., adult-specific promoter). Other variations of GFP are particularly designed for expression in mammalian cells or have up to a 35-fold higher fluorescence. With the aid of a GFP system with a drastically reduced half-life, dynamic processes can also be examined in vivo in the cell.

Literature references relating to A.victoria mutants exhibiting altered fluorescence characteristics include, for example, Heim et al. (1995, Nature 373: 663-664) which relates to mutations at S65 of A. Victoria that enhance fluorescence intensity of the polypeptide. Further references relating to A.victoria mutants include, for example, Ehrig et al., 1995, FEBS Left. 367: 163-166); Surpin et al., 1987, Photochem. Photobiol. 45 (Suppl) : 95S; Delagrave et al., 1995, BioTechnology 13: 151-154; and Yang et al., 1996, Gene 173: 19-23. Patent and patent application references relating to A.victoria GFP and mutants thereof include the following: U.S. Pat. No. 5,874,304 discloses A. Victoria GFP mutants said to alter spectral characteristics and fluorescence intensity of the polypeptide. U.S. Pat. No. 5,968,738 discloses A.victoria GFP mutants said to have altered spectral characteristics. One mutation, V163A, is said to result in increased fluorescence intensity. U.S. Pat. No. 5,804,387 discloses A.victoria mutants said to have increased fluorescence intensity, particularly in response to excitation with 488 nm laser light. U.S. Pat. No. 5,625,048 discloses A.victoria mutants said to have altered spectral characteristics as well as several mutants said to have increased fluorescence intensity. Related U.S. Pat. No. 5,777,079 discloses further combinations of mutations said to provide A.victoria GFP polypeptides with increased fluorescence intensity. International Patent Application (PCT) No. WO 98/21355 discloses A.victoria GFP mutants said to have increased fluorescence intensity, as do WO 97/20078, WO 97/42320 and WO 97/11094. PCT Application No. WO 98/06737 discloses mutants said to have altered spectral characteristics, several of which are said to have increased fluorescence intensity.

The β-glucuronidase (GUS) gene from E. coli can also be operably associated with a C.elegans promoter (e.g., an adult-specific promoter). E.coli GUS has been well documented to provide desirable characteristics as a marker gene in transformed plants. A substrate currently available for histochemical localization of β-glucuronidase activity in tissues and cells is 5-bromo-4-chloro-3-indolyl glucuronide (X-Gluc). The substrate works very well, giving a blue precipitate at the site of enzyme activity. Other substrates include 4-Methylumbelliferyl-β-D-Glucuronide (MUGIcU) which generates a blue product when metabolized and carboxyumbelliferyl-β-D-Glucuronide (CUGIcU) which generates a light blue color when metabolized.

Chitinase production in C.elegans cells worms can be detected by any of several methods known in the art. For example, one method is disclosed by Ellerbrock et al., J. Biomol. Screen 9(2):147-52 (2004): Fluorogenic chitinase substrate (10 μl 0.8 mM of 4-methylumbelliferyl-β-D-N,N′,NΔ-triacetylchito-trioside in DMSO is added to each well of a 96-well plate containing the worms being tested and incubated at 37° C. for 1 hour. The assay is terminated by the addition of 100 μl alkaline buffer (1 M glycine/1 N NaOH, pH 10.6). Wells are read on a fluorimeter at excitation 360/40, emission 460/40, gain 75.

Other reporters include epitope tags that can be expressed directly from a C.elegans promoter (e.g., adult-specific promoter) or appended to an open reading frame that is operably linked to the promoter. Such tags include, for example, glutathione-S-transferase (GST), hexahistidine (His6) tag, maltose binding protein (MBP) tag, haemagglutinin (HA) tag, cellulose binding protein (CBP) tag and myc tag. A convenient method for detecting such a tag is by western blot analysis or by ELISA.

### EXAMPLES

The following example is intended to exemplify and further clarify what is the present invention and should not be construed to limit the present invention. The present invention should not be limited by any mechanism or theory presented herein. Any composition disclosed in the example along with any disclosed method is part of the present invention.

### Example 1

### Genetic Analysis of Human NPC1L1

In this study, we demonstrated that human NPC1L1 can functionally substitute for C.elegans ncr-1 and/or ncr-2. Specifically, we expressed hNPC1L1 from both the ncr-1 and ncr-2 promoters and demonstrated its ability to rescue the dauer-constitutive phenotype of the ncr-1; ncr-2 double mutant.

Materials and Methods

Genetics. Standard methods for handling and genetic manipulation of C. elegans are described by Brenner, S. (Genetics 77: 71-94 (1974)). All experiments were performed at 20° C. Single mutant strains ncr-1(nr2022) and ncr-2(nr2023) and the double mutant strain JT10800 ncr-2(nr2023); ncr-1(nr2022) were used in these experiments. We note that continuous propagation of this strain was reported to result in increasingly subviable animals (Li et al., Development 131: 5741-5752 (2004)). However, under our laboratory conditions we did not observe this phenotype. We also noted that the Daf-C phenotype is completely penetrant under our conditions and is a basis for the rescue assays described in this manuscript.

Plasmid Construction. The plasmid pPD49.26 (Fire et al., Gene 93, 189-198 (1990)) was the starting plasmid for expression vectors designed to express human NPC1L1 from the ncr-1 or ncr-2 promoters. The ncr-1 promoter was PCR amplified from genomic DNA using the primers ncr-14 kbsph5′: GGGGGCATGCCACAACAATTATCTTTATCCTAACT (SEQ ID NO: 11) and ncr1pBam3′: GGGGGGATCCTTCTTGTGCAT CGACTGAAACATACG (SEQ ID NO: 12). Plasmid ncr-1p/hNPC1L1/49.26 contains the 3889 bp ncr-1 promoter inserted into the Bam HI and Nhe I restriction sites of pPD49.26 and human NPC1L1 (Altmann et al., Science 303, 1201-4 (2004)) inserted into the Nhe I and Kpn I restriction sites of pPD 49.26.

The ncr-2 promoter was PCR amplified from genomic DNA using the primers ncrp2245: CTATACATTTATGCCTCAGAGCAATCA (SEQ ID NO: 13) and ncr-23′prom: TCCGGAAATGTAGAAATTTAATATTAAATACT (SEQ ID NO: 14). Plasmid ncr-2p/hNPC1L1/49.26 contains the 4198 bp ncr-2 promoter inserted into the Sma I restriction site of pPD49.26 and human NPC1L1 (Altmann et al., Science 303, 1201-4 (2004)) inserted into the Nhe I and Kpn I restriction sites of pPD49.26.

GFP was amplified from pPD95.67 (Fire et al., Gene 93, 189-198 (1990)) using primers GFP5′Xba: GGGGTCTAGAATGAGTAAAGGAGAAGAACTTTTCACTG (SEQ ID NO: 15) and GFP3′Not: CCCCGCGGCCGCCTATTTGTATAGTTCATCCATGCC ATGTGT (SEQ ID NO: 16). The ends of this GFP PCR fragment were made blunt using Klenow enzyme. This GFP PCR fragment was inserted into the blunt site generated from the removal of human NPC1L1 from ncr-1p/hNPC1L1/49.26 by digestion with restriction enzymes Nhe I and Eco RV, followed by treatment with Klenow enzyme. The resulting plasmid was called ncr-1p/GFP/49.26. In addition, this GFP PCR fragment was inserted into the blunt restriction site generated from the removal of human NPC1L1 from ncr-2p/hNPC1L1/49.26 by digestion with Nhe I and Kpn I, followed by treatment with Klenow enzyme. The resulting plasmid was called ncr-2p/GFP/49.26. The expression patterns from both the ncr-1 and ncr-2 promoters have been described previously (Li et al., Development 131, 5741-5752 (2004)). We noted the following differences between the expression pattern they describe from the ncr-2 promoter and the one that we observe. First, our ncr-2p/GFP/49.26 is not expressed in the gonadal sheath as shown by Li et al. (2004). Second, we observed expression throughout the ventral nerve cord in most animals. We assessed our expression pattern in both a wild-type background and an ncr-2(nr2023); ncr-1(nr2022) mutant background and saw no difference between the strains.

A genomic fragment encompassing the ncr-1 genomic coding region was PCR amplified using the primers ncr5′sma: CCCGGGAAACAACTACTCATTTTTTGC (SEQ ID NO: 17) and ncr13′A: GATTTATGTGTTCTACTTATGTTC (SEQ ID NO: 18). The resulting PCR product was 8338 bp long and began just after the ATG and ended 996 bp after the stop codon. Thus, this resulted in an ncr-1 genomic fragment where the ATG is deleted and is replaced by a Sma I restriction site. This PCR fragment was directly inserted into the pCRXL vector (Invitrogen; Carlsbad, Calif.).

A 7.3 kb genomic fragment encompassing the ncr-2 genomic coding region but starting just 3′ of the ATG was PCR amplified from N2 genomic DNA using the primers ncr-2noATG: CGTCAAGGAGGAGGAGGAGGCGAG (SEQ ID NO: 19) and ncr-23′UTR: CTGAAATCGGATAAATAAATTAATAAAT (SEQ ID NO: 20). This PCR fragment was directly inserted into the pTOPO XL vector (Invitrogen; Carlsbad, Calif.).

The ncr-1 1st intron was PCR amplified as a 1.047 kb fragment with the

| | |
|---|---|
| primers ncr-13′ intron1: | |
| CGATACTAATGTGGAGCCCACAGC | (SEQ ID NO:21) |
| and | |
| ncr-15′ intron1: | |
| CGAAGCACGACGGACATCGTCCCAG. | (SEQ ID NO:22) |

This PCR fragment was directly inserted into pCR4 TOPO vector (Invitrogen; Carlsbad, Calif.).

The ncr-2 14th intron was PCR amplified as a 3.5 kb fragment from N2 genomic DNA using the primers

| | |
|---|---|
| F09G8F2438: | |
| GATTGGTTCAAGCTTGCGATCGC | (SEQ ID NO:23) |
| and | |
| F09G8R5969: | |
| GAGCACATTGGATTGATGGAGGAGTCTC. | (SEQ ID NO:24) |

This PCR fragment was directly inserted into the pCR4 TOPO vector (Invitrogen; Carlsbad, Calif.). Intron 14 of ncr-2 contained the coding region of the col-91 gene; preliminary data suggested that expression of this gene was hindering our ability to recover transgenic animals. Therefore, we engineered a frameshift in the 2nd exon of col-91 by digesting with BsmB1 and treating with T4 polymerase. Sequence analysis of the resulting fragment showed a 4 bp deletion, resulting in a frameshift within the coding region of this gene. All transgenic animals described in this work containing the ncr-2 14^{th} intron contain this form with the frameshift within col-91.

Microinjection. All plasmids were injected at 20 ug/ml along with the dominant rol-6 co-transformation marker pRF4 at 100 ug/ml (Mello et al., EMBO J. 10, 3959-3970 (1991)) into the double mutant strain ncr-2(nr2023); ncr-1(nr2022), unless noted otherwise. Lines were established and maintained while the animals were grown on standard NGM plates, which included the addition of cholesterol at a final concentration of 5 ug/ml (Brenner, Genetics 77: 71-94 (1974)).

Rescue assays. Adult, transgenic animals were transferred to NGM-plates, which were similar to the standard NGM plates except no exogenous cholesterol was added. These plates still had residual levels of cholesterol originating from the agar and peptone components of the media. Under these conditions, the ncr-2(nr2023); ncr-1(nr2022) double mutant did not exit the dauer stage readily. This is in contrast to double mutant animals grown on standard NGM plates where they entered the dauer stage only transiently; after a day or so, the dauers recoverd and continued normal development. Adult, transgenic animals were allowed to lay eggs on NGM-plates for 2-3 hours and then removed. Roller progeny on these plates were scored 75-120 hours later for progression through the L3 stage to the adult stage.

Indirect Immunofluorescence. Animals were fixed as described in Bettinger et al. (1996). A 1:200 dilution of polyclonal antibody A1801 (Iyer et al., Biochim Biophys Acta. 1722: 282-292 (2005)), which recognizes rat and human NPC1L1 was pre-absorbed against fixed, ncr-2(nr2023); ncr-1(nr2022) double mutant worms overnight at 4° C. The supernatant was removed and added to fixed, transgenic animals or fixed double mutant animals as a control. Worms were washed, incubated with Cy3-conjugated secondary antibody and washed again as described in Levitan and Greenwald, Development 125:3101-3109 (1998) and viewed with a Zeiss Axiphot 2 MOT microscope equipped with an MTI CCD camera.

Results

To determine if human NPC1L1 can functionally substitute for ncr-1 and/or ncr-2, we expressed human NPC1L1 from the ncr-1 promoter. Rescue was determined by making transgenic lines in an ncr-2; ncr-1 mutant background. To make transgenic lines, the test plasmid was co-injected with a visible co-transformation marker, in this case the plasmid pRF4 (Mello et al., EMBO J. 10: 3959-3970 (1991)) which are then assembled into a single, extrachromosomal array following injection into the animal. Animals bearing these extrachromosomal arrays were selected by visible inspection and transgenic lines were established.

We were then able to assess the ability of test plasmids to rescue the ncr-2; ncr-1 constitutive dauer phenotype. ncr-2; ncr-1 mutant animals grown on standard (NGM) media constitutively entered the dauer stage only transiently; within 2-4 days they recovered and grew to adulthood (Sym et al., Current Biology 10: 527-530 (2000)). However, if we reduced cholesterol in the media, as we did in NGM-media (see Materials and Methods), ncr-2; ncr-1 mutant animals either arrested as dauer or stayed in the dauer stage significantly longer than those grown on standard NGM media.

We first tested the plasmids ncr-1p/hNPC1L1/49.26 or ncr-2p/hNPC1L1/49.26 individually or together by making transgenic animals bearing these plasmids in an ncr-2 ncr-1 mutant background. Two lines were generated by injecting ncr-2p/hNPC1L1/49.26 at 100 ug/ml and five lines were generated by co-injection of the two plasmids ncr-1p/hNPC1L1/49.26+ncr-2p/hNPC1L1/49.26, at 100 ug/ml each. None of these lines demonstrated any ability to rescue the dauer-constitutive phenotype of ncr-2; ncr-1 mutant animals.

Proper expression of many genes in C.elegans is only accomplished by the inclusion of genomic regulatory elements in addition to the 5′ promoter region. These regulatory elements are often found in introns and must be included to achieve full expression of a given gene (see for example, Struhl et al., Cell 74: 331-345 (1993) and Levitan et al., Proc. Natl. Acad. Sci. U.S.A. 98: 12186-12190 (2001)). To that end, we PCR amplified the 8 kb genomic region from the ncr-1 gene, encompassing the entire genomic region beginning just after the ATG to the 3′ UTR. Our 5′ primer was designed in such a way so that, upon amplification, a unique Sma I restriction enzyme site was inserted at the site of the initiating ATG (see Materials and Methods). This PCR fragment was directly cloned into the TOPO (Invitrogen, Inc) vector system.

We co-injected the ncr-1 8 kb genomic region with the plasmids ncr-1 p/hNPC1L1/49.26 and ncr-2p/hNPC1L1/49.26 into the ncr-2; ncr-1 double mutant. Since co-injection of plasmids results in the random assembly of large extrachromosomal arrays, we reasoned that the regulatory regions contained on the 8 kb ncr-1 genomic fragment would, by chance, reside in close proximity to the plasmids expressing hNPC1L1 and would enhance expression from those plasmids. As a control, we co-injected the 8 kb ncr-1 genomic fragment with ncr-1p/GFP/49.26. The results of these experiments are shown in Table 1.

TABLE 1
Assessment of rescue activity in arrays carrying
the 8 kb ncr-1 genomic fragment.^{∝}

| Transgene | | % Rescue (n) | |
|---|---|---|---|
| 1) none | | 0.04 | (421) |
| 2) ncr-1p::GFP | line 1 | 2.30 | (296) |
| | line 2 | 2.40 | (289) |
| 3) ncr-2p::GFP | line 1 | 2.90 | (140) |
| | line 2 | 4.80 | (188) |
| | line 3 | 1.50 | (167) |
| 4) ncr-2p::hNPC1L1 | line 1 | 74.30 | (183) |
| | line 2 | 24.10 | (83) |
| | line 3 | 61.50 | (127) |
| 5) ncr1p::hNPC1L1 + | line 1 | 40.10 | (237) |
| ncr2p::hNPC1L1 | line 2 | 51.70 | (207) |
| ^{∝}All animals are in an ncr-2(nr2023); ncr-1(nr2022) background. Transgenic animals or control animals (labeled “none”) were transferred to NGM- plates and allowed to lay eggs for 2-3 hours, then removed. Eggs were grown on NGM-plates and scored 72-120 hours after being laid. Only animals carrying the visible, co-transformation | | | |
| # marker pRF4 were scored for whether they progressed through the L3 stage and reached L4 or adulthood. The number in parenthesis is the number of animals scored. Results of this representative experiment are an average of three separate plates for each condition. All arrays also contain the 8 kb ncr-1 genomic fragment. | | | |

Injection of the 8 kb ncr-1 genomic fragment with only the co-transformation marker resulted in no discernible rescue of the ncr-2(nr2023); ncr-1(nr2022) Daf-c phenotype (data not shown). This result indicated that the 8 kb ncr-1 genomic fragment had no inherent rescuing activity. Transgenic animals carrying the 8 kb ncr-1 genomic fragment with the plasmid expressing GFP from the ncr-1 promoter (ncr-1p/GFP/49.26) or the ncr-2 promoter (ncr-2p/GFP/49.26) were also not rescued (Table 1). This result demonstrated that the 8 kb ncr-1 genomic fragment did not undergo some aberrant recombination events during extrachromosomal array formation that would generate an intact ncr-1 gene (the ncr-1 or ncr-2 promoter driving expression from the ncr-1 genomic fragment). Finally, 40-50% of the transgenic animals expressing human NPC1L1 from the ncr-1 and ncr-2 promoters did not form dauers constitutively-they progressed through development without arresting at the dauer stage. In addition, 24-75% of the transgenic animals expressing hNPC1L1 from only the ncr-2 promoter exhibited rescue. These results indicated that human NPC1L1 can functionally substitute for ncr-1 and/or ncr-2.

To confirm that the rescue we observed is a result of hNPC1L1 protein expression, we first integrated an extrachromosomal array containing ncr-1p/hNPC1L1/49.26, ncr-2p/hNPC1L1/49.26 and the 8 kb ncr-1 genomic fragment into a chromosome so that every cell in the animal would carry a stable form of these transgenes. We then used an antibody directed against rat NPC1L1 that also cross-reacts with the human protein, A1801 (Iyer et al., Biochim Biophys Acta. 1722: 282-292 (2005)), to detect protein in fixed worms by indirect immunofluorescence. In these studies, all animals were in an ncr-2(nr2023); ncr-1(nr2022) background. These animals carried an extrachromosomal array carrying the ncr-1p/hNPC1L1/49.26, ncr-2p/hNPC1L1/49.26 and the 8 kb ncr-1 genomic fragment constructs. We detected specific expression in a pair of neuron-like cells in the head, between the anterior and posterior bulb of the pharynx. This expression is seen only in those two cells and only in animals that are younger than the L3 stage. Interestingly, this temporal and spatial restriction corresponds to expression seen from the daf-9 promoter, which was shown to be restricted to the XXX cell (Jia et al., Development 129: 221-231 (2002)). This pair of cells has been demonstrated to be critical for the constitutive dauer phenotype-laser ablation of the XXX cells in a wild-type animal induces dauer formation (Gerisch et al., Dev Cell 1: 841-851 (2001)).

The results shown in table 1 suggested that the 8 kb ncr-1 genomic fragment, while possessing no intrinsic rescuing activity, can provide regulatory regions that augment expression or activity from the ncr-1p/hNPC1L1 and ncr-2p/hNPC1L1 constructs. We next wanted to assess whether the ncr-2 genomic region also had this ability to confer rescuing activity when co-injected with hNPC1L1 expression vectors. For simplicity, we chose to focus on the ncr-2p/hNPC1L1/49.26 expression construct, since it demonstrated ample rescuing activity in coinjection experiments with the ncr-1 genomic region.

We made the identical construct for ncr-2, where we PCR amplified the 7.3 kb ncr-2 genomic region from one codon downstream of the initiating ATG to the stop codon (see Materials and Methods). As shown in table 2 (infra), when this ncr-2 genomic region was coinjected with ncr-2p/GFP/49.26, no rescuing activity was observed. This result suggested that this ncr-2 genomic fragment, like its ncr-1 counterpart, has no intrinsic rescuing activity. However, when we coinject the ncr-2 7.3 kb genomic fragment with ncr-2p/hNPC1L1/49.26, we observed that 18-39% of the transgenic animals were rescued. These results suggested that the ncr-2 genomic fragment also contained information that can augment expression or activity from the ncr-2 promoter.

TABLE 2
Assessment of rescue activity in arrays carrying
the 7.3 kb ncr-2 genomic fragment.^{∇}

| Transgene | | % Rescue | |
|---|---|---|---|
| 1) none | | 0.0 | (169) |
| 2) ncr-2p::GFP | line 1 | 3.5 | (141) |
| | line 2 | 6.4 | (110) |
| 3) ncr-2p::hNPC1L1 | line 1 | 36.2 | (207) |
| | line 2 | 18.4 | (136) |
| | line 3 | 39.9 | (188) |

^{∇}All animals were in an ncr-2(nr2023); ncr-1(nr2022) background. Details of the experiment were essentially identical to that described in table 1 legend except that all arrays contained the 7.3 kb ncr-2 genomic fragment.

We attempted to visualize this enhancement of expression by comparing GFP expression from an extrachromosomal array carrying the ncr-2p/GFP/49.26 construct with an extrachromosomal array carrying the ncr-2p/GFP/49.26 construct coinjected with the 8 kb ncr-1 genomic region. While expression from the latter array seemed qualitatively stronger (data not shown), we found it difficult to quantify this difference due to the overall weak signal generated from these arrays.

Since both the entire ncr-1 and ncr-2 genomic regions can augment rescue of the Daf-C phenotype when hNPC1L1 is expressed from either the ncr-1 or ncr-2 promoter, we tried to identify more specific sequences within these genomic regions that may be playing an important role in this process. Introns in C. elegans are typically small, with a median size of 65 nucleotides (Spieth et al., J. and Lawson, D. (2005). Overview of Gene Structure. In Wormbook, vol. 2005 (ed.: The C. elegans Research Community, WormBook, doi/10.1895/wormbook.1.7.1). We reasoned that the larger introns are more likely to contain regulatory information, as demonstrated by the 1.5 kb intron in the gene pal-1 that was shown to contain regulatory information (Zhang et al., Genes Dev. 14: 2161-2172 (2000)). We chose the first intron of ncr-1 (861 bp) and the 14^{th} intron of ncr-2 (1.8 kb) for further study. Again, we focused our analysis on the ncr-2p/hNPC1L1/49.26 expression vector. As shown in table 3 (infra), the first intron from ncr-1 or the 14^{th} intron from ncr-2 were able to augment rescuing activity of hNPC1L1 from the ncr-2 promoter. This suggested that these introns may contain regulatory information that enhances activity from the ncr-2 promoter in the proper cell types.

TABLE 3
Assessment of rescue activity in arrays carrying the first
intron from ncr-1 or the fourteenth intron from ncr-2.^{±}

| Transgene | | % Rescue (n) | |
|---|---|---|---|
| 1) none | | 0.92 | (216) |
| 2) ncr-2p::hNPC1L1 + | line 1 | 24.70 | (207) |
| ncr-1 intron 1 | line 2 | 22.60 | (115) |
| | line 3 | 16.20 | (204) |
| 3) ncr-2p::hNPC1L1 + | line 1 | 17.90 | (184) |
| ncr-2 exon 14 | line 2 | 10.60 | (141) |
| | line 3 | 10.10 | (144) |

^{±}Details of the experiment are essentially identical to that described in table 1 legend except that all arrays contain only the transgenes indicated in the table.

Discussion

We have demonstrated, in this example, that human NPC1L1 can functionally substitute for ncr-1 and/or ncr-2. Although the degree of homology between ncr-1/ncr-2 and human NPC1L1 is around 30%, there is precedent for this kind of functional conservation between worm and human genes with this low degree of identity (for example, see Wu et al., Nature 392: 501-504 (1998)). While human NPC1L1 has been implicated in cholesterol absorption in the intestine, currently there is no functional assay for this protein. Data presented herein provide the first description of a functional assay for human NPC1L1.

We have shown that the genomic regions of the ncr-1 and ncr-2 genes provide some regulatory information in this assay, thus enabling us to demonstrate rescue by the hNPC1L1 gene. We have shown that the hNPC1L1 protein is in fact expressed in a relevant temporal and spatial pattern and that at least some of this regulatory information can be found in the large introns of the ncr genes. It is unlikely the two introns tested here contain all the regulatory information, since the level of rescue is considerably less than that observed with the entire ncr-1 or ncr-2 genomic regions. Furthermore, comparison of the ncr-1 intron 1 and ncr-2 intron 14 sequences with the corresponding introns from the related nematode C. briggsae, revealed no substantial stretches of homology. This suggests any regulatory information located in these introns may be diffuse.

There are several uses for a functional assay for human NPC1L1 in C. elegans. It enables us to perform detailed structure/function analysis of the protein. For example, we can elucidate which parts of the protein, like specific transmembrane domains or the sterol-sensing domain are critical for its function. In addition, we can examine what effect single nucleotide polymorphisms (SNPs) that are found in the human population have on the function of this protein.

Furthermore, transgenic C. elegans expressing human NPC1L1 can be used in a screening assay to identify compounds that inhibit the function of this protein. Such compounds have profound effects on cholesterol absorption in mammals. Human NPC1L1 can rescue the Daf-c phenotype of ncr-2; ncr-1 mutant animals, thus allowing them to progress to adulthood. We could employ a high-throughput assay taking advantage of this phenotype. For example, adult animals make eggs which secrete chitinase into the media to enable the animal to hatch out of the egg shell. Compounds that inhibit human NPC1L1 would result in the lack of progression to the adult stage. These animals would secrete significantly less chitinase into the media as compared to transgenic animals where human NPC1L1 was not inhibited. An enzymatic assay utilizing chitinase activity in C. elegans has already been described as a means to look for compounds that affect the function of the worm homolog of human presenilin (Ellerbrock et al., J Biomol Screen. 9: 147-152 (2004)). Alternatively, GFP could be expressed from an adult-specific promoter such as col-19 (Liu et al., Development: 121: 2471-2478 (1995)) which could be monitored to assess progression through the adult stage and the inhibition of this process by compounds.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, accession numbers, publications, product descriptions, and protocols are cited throughout this application, the disclosures of which are incorporated herein by reference in their entireties for all purposes.

We claim:

1. A method for identifying a substance that inhibits intestinal cholesterol absorption, reduces elevated total cholesterol, reduces elevated low density lipoprotein cholesterol, reduces elevated apolipoprotein B, treats or prevents heterozygous familial hypercholesterolemia, treats or prevents non-familial hypercholesterolemia, treats or prevents homozygous familial hypercholesterolemia, or treats or prevents homozygous sitosterolemia comprising:
(a) contacting a C.elegans worm having a functional NPC1L1 polypeptide but lacking functional ncr-1 and ncr-2 polypeptide with the substance to be tested; and
(b) determining if the worm exhibits a dauer phenotype;
whereby the substance is identified as being an inhibitor of intestinal cholesterol absorption, capable of reducing elevated total cholesterol, capable or reducing elevated low density lipoprotein cholesterol, capable of reducing elevated apolipoprotein B, useful for treating or preventing heterozygous familial hypercholesterolemia, useful for treating or preventing non-familial hypercholesterolemia, useful for treating or preventing homozygous familial hypercholesterolemia, or useful for treating or preventing homozygous sitosterolemia if the dauer phenotype is observed.

2. The method of claim 1 wherein the phenotype is identified by visual inspection.

3. The method of claim 1 wherein the NPC1L1 is human NPC1L1.

4. The method of claim 3 wherein the human NPC1L1 comprises the amino acid sequence set forth in SEQ ID NO: 6.

5. A method for identifying a substance that inhibits intestinal cholesterol absorption, reduces elevated total cholesterol, reduces elevated low density lipoprotein cholesterol, reduces elevated apolipoprotein B, treats or prevents heterozygous familial hypercholesterolemia, treats or prevents non-familial hypercholesterolemia, treats or prevents homozygous familial hypercholesterolemia, or treats or prevents homozygous sitosterolemia comprising:
(a) contacting a C.elegans worm having a functional NPC1L1 polypeptide but lacking functional ncr-1 and ncr-2 polypeptide with the substance to be tested; and
(b) determining whether the worm secretes chitinase;
whereby the substance is identified as being an inhibitor of intestinal cholesterol absorption, capable of reducing elevated total cholesterol, capable of reducing elevated low density lipoprotein cholesterol, capable of reducing elevated apolipoprotein B, useful for treating or preventing heterozygous familial hypercholesterolemia, useful for treating or preventing non-familial hypercholesterolemia, useful for treating or preventing homozygous familial hypercholesterolemia, or useful for treating or preventing homozygous sitosterolemia if chitinase is not secreted.

6. The method of claim 5 wherein chitinase is detected by measuring cleavage of the substrate 4-methylumbelliferyl-β-D-N,N′,N″-triacetylchito-trioside.

7. The method of claim 5 wherein the NPC1L1 is human NPC1L1.

8. The method of claim 7 wherein the human NPC1L1 comprises the amino acid sequence set forth in SEQ ID NO: 6.

9. A method for identifying a substance that inhibits intestinal cholesterol absorption, reduces elevated total cholesterol, reduces elevated low density lipoprotein cholesterol, reduces elevated apolipoprotein B, treats or prevents heterozygous familial hypercholesterolemia, treats or prevents non-familial hypercholesterolemia, treats or prevents homozygous familial hypercholesterolemia, or treats or prevents homozygous sitosterolemia comprising:
(a) contacting a C.elegans cell having a functional NPC1L1 polypeptide and having an adult-specific C.elegans promoter operably linked to a reporter but lacking functional ncr-1 and ncr-2 polypeptide with the substance to be tested; and
(b) determining whether expression by the promoter occurred;
whereby the substance is identified as being an inhibitor of intestinal cholesterol absorption, capable of reducing elevated total cholesterol, capable of reducing elevated low density lipoprotein cholesterol, capable of reducing elevated apolipoprotein B, useful for treating or preventing heterozygous familial hypercholesterolemia, useful for treating or preventing non-familial hypercholesterolemia, useful for treating or preventing homozygous familial hypercholesterolemia, or useful for treating or preventing homozygous sitosterolemia if the expression is not detected.

10. The method of claim 9 wherein the NPC1L1 is human NPC1L1.

11. The method of claim 10 wherein the human NPC1L1 comprises the amino acid sequence set forth in SEQ ID NO: 6.

12. The method of claim 9 wherein the adult-specific C.elegans promoter is a member selected from the group consisting of the col-19 promoter and the vit-2 promoter.

13. The method of claim 9 wherein the reporter is a member selected from the group consisting of Photorhabdus luminescens LuxA-E, FMN oxidoredtuctase; amFP486; zFP506; zFP538; dsFP483; drFP583; cFP484; Pyrophorus plagiophthalamus luciferase; Chloramphenicol Acetyltransferase (CAT); β-Galactosidase (β-Gal); Vibrio harveyi luciferase; Photinus pyralis Luciferase; Renilla reniformis luciferase; Green Fluorescent Protein; β-glucuronidase (GUS) and chitinase.

14. A method for producing NPC1L1 comprising introducing a polynucleotide encoding NPC1L1 operably linked to a promoter into a C.elegans cell and propagating said cell.

15. The method of claim 14 wherein the NPC1L1 is isolated from the propagated cell.

16. An isolated transgenic Caenorhabditis elegans worm whose cells lack functional ncr-1 protein and ncr-2 protein and have functional NPC1L1 protein.

17. The worm of claim 16 which is strain N2 having a functional NPC1L1 polypeptide but lacking functional ncr-1 and ncr-2 polypeptide.

18. The worm of claim 16 wherein the NPC1L1 is human NPC1L1.

19. The worm of claim 18 wherein the human NPC1L1 comprises the amino acid sequence set forth in SEQ ID NO: 6.

20. The worm of claim 16 whose cells comprise NPC1L1 polynucleotide integrated into a C.elegans chromosome.

21. The worm of claim 16 whose cells comprise a polynucleotide encoding functional NPC1L1 which is operably associated with a C.elegans promoter.

22. The worm of claim 21 wherein the promoter is selected from the group consisting of the ncr-1 promoter and the ncr-2 promoter.

23. An isolated transgenic C.elegans worm that is selected from the group consisting of Strain 2a, Strain 2b, Strain 3, Strain 4a and Strain 4b.

24. An isolated transgenic C.elegans worm whose cells comprise functional NPC1L1 polypeptide.

25. An isolated plasmid selected from the group consisting of ncr-1p/hNPC1L1/49.26; ncr-2p/hNPC1L1/49.26 and ncr-1p/GFP/49.26.

26. An isolated oligonucleotide selected from the group consisting of SEQ ID NOs: 7-14.


SEQUENCE LISTING (24 sequences)

SEQ ID NO: 1 - DNA, 4152 bp, Caenorhabditis elegans
  CDS (1)..(4152)
atgaaacaactactcattttttgcttgctatttgggtctatattccatcatggcgacgcgggatgtatcatgcgaggattgtgccagaagcatactgaaaatgcatatggaccatgtgttaccaacgatactaatgtggagcccacagcttttgacaaaactcatccggcatatgagaaaatggtcgagttttgcccccatttgctaactggtgacaacaaactctgctgtacgccatcgcaagcggaaggactgactaagcaaattgcacaagcccgacatattctgggacgatgtccgtcgtgcttcgacaactttgccaaattgtggtgcgaatttacgtgcagcccgaatcaacaggattttgtatcgatctccgaaatgaaaccaatcgagaagaaagagggattcaccccagagtaccaaccagctgaggcatacgtgaatacagtcgagtacagattgtctactgattttgctgagggaatgttctcgtcttgtaaagatgtaacttttggagggcagcctgcattacgagtcatgtgtacttctactccgtgtactttgacaaattggttggaatttattggaactcaaaatctagatctcaatattccaatccacacaaagttccttctctatgaccctatcaagacacctccatcggatcgttcaacgtacatgaatgtcaactttactgggtgcgacaaatcagctcgagtcggctggccggcttgttcaacgtcagagtgcaacaaggaagagtacgcaaacttgatcgacctggacgatggaaaaacttctgggcaaacttgcaatgttcatggcatcgcctgcctgaacatttttgtcatgctcgccttcatcggctcacttgccgtgcttctttgtgttggatttgtattcacatcatatgacgaagactacacgaacttgaggcaaacacaatctggagaagaatccccgaaacggaacagaatcaaacggaccggtgcctggattcacaacttcatggagaacaatgctcgagatattggaatgatggcgggacgcaatccaaagtctcactttttcataggatgtgccgttctgattttctgcttgccaggaatgatttatcacaaagagtcgacaaatgttgttgatatgtggtcttctccgagatccagggctcgacaagaagagatggttttcaatgccaactttggacgcccacagagatatcagcaaattatgttgctaagtcaccgcgatttccaatcgagcgggaagctttatggaccagtattccacaaggacatttttgaggagcttttcgatattttaaatgccatcaaaaatatatcaacgcaagactctgatggtcggacaatcacacttgatgatgtatgttatcgacctatgggaccaggatatgattgtttgattatgtctccaacgaactattttcaagggaacaaagagcatctggatatgaaatcgaacaaagaagaaacggtttccgaagatgatgacgcgtttgattatttctcatcggaagcaacaacagatgaatggatgaatcacatggcagcatgtatagatcaaccaatgtcccaaaaaacaaagtccggcctgagctgcatgggaacatacggtggaccatctgctcctaacatggtcttcggcaaaaatagtacgaatcatcaagctgccaactcgatcatgatgactatcctggtcacacagagaactgagccggagattcaaaaagcagaactttgggagaaggagtttttgaagttctgcaaagagtacagagaaaagtctccgaaagtgattttctcgtttatggcagaaaggtcgatcactgatgagattgaaaacgacgcaaaggatgaaattgtgactgttgtgattgctctggcttttctcatcggttatgttacattctctctcgggcgttactttgtgtgtgagaatcagctttggtcaattctggtacattctcgaatctgtcttggaatgctgagcgtcatcataaacttgctcagttcgttctgctcgtggggaattttctccatgtttggaatccatccggtcaaaaatgcgttggtcgtacaattcttcgttgtgacgttgctaggcgtctgtcgtacattcatggttgttaagtactacgctcaacaaagagtctccatgccttacatgtcccccgaccaatgtccagagatcgtgggaatggtcatggctggaactatgccagctatgttcagtagctcgttgggatgtgctttcagctttttcattggtggattcacagatttgcctgcaatccgaacattttgtctctacgccggtcttgctgttcttattgatgtggtcttacattgcacaattttccttgcacttttcgtatgggacactcaaagggagctcaatggaaaaccagagttctttttcccatatcaaatcaaggatcttttgggtgcttaccttattggtcgtcagagggcgacagacacttttatgacacaattcttccactttcaagtagctccgttcctgatgcacagaatgactcgtattatcacaggaataatcttcattgcttctttcattacaacagtgatcctctcaagtaagatcagcgttggattcgaccagagtatggctttcacagagaaaagctacatcagcacgcactttcgatatctcgacaaattttttgacgtgggaccgccagtgtttttcactgttgacggagaacttgattggcacagaccagatgttcagaacaagttttgtacatttcctgggtgtagcgacacttcgtttggaaacattatgaattacgcagttggacacacggagcagacgtacttgtccggtgaaatgtataactggattgacaactatctggaatggatttcaagaaaaagcccatgttgtaaagtttatgtgcacgaccccaacacattctgctcgacaaaccggaacaagagtgcgttagacgataaggcatgtagaacttgcatggactttgattacgtggccaactcgtacccgaaaagctcaatcatgtaccatcgtccgtcaatcgaagttttctaccgtcatctgagacactttttagaagacactccaaattctgaatgcgttttcggtggccgtgcttcattcaaagatgcaatcagcttcacatctagaggacgtattcaagcatctcaattcatgactttccacaaaaagctgtcaattagcaactccagtgatttcatcaaggcaatggacactgctcgaatggtatcccgtcgccttgaaagatcaattgacgatacagctcacgtcttcgcatacagcaaaattttcccattctacgaacagtactctactatcatgccaattctcactacccaactttttatcactgttgttggcgtcttcggcatcatttgtgttactcttgggattgatgtgaaaggagcggcttgcgctgtaatttgtcaagtgtccaactacttccatattgtggcatttatgtatatctttaatatccccgtcaatgccttgtctgcaacaaatcttgtaatgtcgtctggaattctaatcgagttttctgtaaatgttctcaaaggatacgcctgctcacttcgccagcgagcaaaagatcgcgcagagtctacggttggaagtattggaccaatcattctctccggcccagttgtcactatggctggatctacaatgtttctctcgggggctcatcttcagattataacggtctactttttcaaattgttcctcatcactattgtcagcagtgctgtccatgctctcatcatattgcctattttgcttgccttcggaggatcgagaggccatggaagttcagagactagcacgaatgacaatgacgagcaacatgacgcgtgcgttttgtcgccaaccgcggaaagtcatatttcaaacgtggaagaaggaatccttaaccgtccttctttactggacgccagccacattctcgatccgcttcttaaagcggaaggtgggattgataaagcaattgatatcataacaatcgatcgctcttacccatctacgccatcttcactgccatgtacctcccgaatgccaagagctcatatagaaccggatctccgatctttgtag

SEQ ID NO: 2 - protein, 1383 aa, Caenorhabditis elegans
MKQLLIFCLLFGSIFHHGDAGCIMRGLCQKHTENAYGPCVTNDTNVEPTAFDKTHPAYEKMVEFCPHLLTGDNKLCCTPSQAEGLTKQIAQARHILGRCPSCFDNFAKLWCEFTCSPNQQDFVSISEMKPIEKKEGFTPEYQPAEAYVNTVEYRLSTDFAEGMFSSCKDVTFGGQPALRVMCTSTPCTLTNWLEFIGTQNLDLNIPIHTKFLLYDPIKTPPSDRSTYMNVNFTGCDKSARVGWPACSTSECNKEEYANLIDLDDGKTSGQTCNVHGIACLNIFVMLAFIGSLAVLLCVGFVFTSYDEDYTNLRQTQSGEESPKRNRIKRTGAWIHNFMENNARDIGMMAGRNPKSHFFIGCAVLIFCLPGMIYHKESTNVVDMWSSPRSRARQEEMVFNANFGRPQRYQQIMLLSHRDFQSSGKLYGPVFHKDIFEELFDILNAIKNISTQDSDGRTITLDDVCYRPMGPGYDCLIMSPTNYFQGNKEHLDMKSNKEETVSEDDDAFDYFSSEATTDEWMNHMAACIDQPMSQKTKSGLSCMGTYGGPSAPNMVFGKNSTNHQAANSIMMTILVTQRTEPEIQKAELWEKEFLKFCKEYREKSPKVIFSFMAERSITDEIENDAKDEIVTVVIALAFLIGYVTFSLGRYFVCENQLWSILVHSRICLGMLSVIINLLSSFCSWGIFSMFGIHPVKNALVVQFFVVTLLGVCRTFMVVKYYAQQRVSMPYMSPDQCPEIVGMVMAGTMPAMFSSSLGCAFSFFIGGFTDLPAIRTFCLYAGLAVLIDVVLHCTIFLALFVWDTQRELNGKPEFFFPYQIKDLLGAYLIGRQRATDTFMTQFFHFQVAPFLMHRMTRIITGIIFIASFITTVILSSKISVGFDQSMAFTEKSYISTHFRYLDKFFDVGPPVFFTVDGELDWHRPDVQNKFCTFPGCSDTSFGNIMNYAVGHTEQTYLSGEMYNWIDNYLEWISRKSPCCKVYVHDPNTFCSTNRNKSALDDKACRTCMDFDYVANSYPKSSIMYHRPSIEVFYRHLRHFLEDTPNSECVFGGRASFKDAISFTSRGRIQASQFMTFHKKLSISNSSDFIKAMDTARMVSRRLERSIDDTAHVFAYSKIFPFYEQYSTIMPILTTQLFITVVGVFGIICVTLGIDVKGAACAVICQVSNYFHIVAFMYIFNIPVNALSATNLVMSSGILIEFSVNVLKGYACSLRQRAKDRAESTVGSIGPIILSGPVVTMAGSTMFLSGAHLQIITVYFFKLFLITIVSSAVHALIILPILLAFGGSRGHGSSETSTNDNDEQHDACVLSPTAESHISNVEEGILNRPSLLDASHILDPLLKAEGGIDKAIDIITIDRSYPSTPSSLPCTSRMPRAHIEPDLRSL

SEQ ID NO: 3 - DNA, 3825 bp, Caenorhabditis elegans
  CDS (1)..(3825)
atgcgtcaaggaggaggaggaggcgagagaatggtatctgtcctattcttattgctaatacatttggcattgtgccaagcaaaatgtgtgatgacggaatgtgacggagaggaggatagcaaccatccaccatgcaagactaacaagtcaacatatctaccaatcaccgtgacacggtctctaaatccaacttatatggctcgattcgaaaagtactgctcgtatcttgtacaggaagaggataaagctcaagtctgctgtacagaactgcaattaaaaggaatgactgatcgaatttctaatgctgctacaatccttggatcttgtcccagttgcttcgacaattttgcaaaactctggtgtcaattcacttgttccccagatcaatcaaaattcatgaaggtaatggaaacgactggtccgaaaaatgtcgtggttaaaatggagttcaaagtgaatcgagattttgtggagggattgtacgagtcatgccgtcatacgtggtttgccaatggacttgctcttcgattgatgtctcttggtggaaaagtctcttttgaaaacttttatggatttatgggaacgaagaatcttgcacagagtattccaataaacaccgagtttcagttttctcgaatgaaaaacgcaatgaatattccaacaacaccatgtcacaaatcagctggccccaaagttccagcttgtggagctattgattgtccaacaaatgcccatcaacttgtagatatctccaaagttgaacatctcggaacgaaagtgttccatcctcacttcccagactttgaatggttactgaaaatctgcggatgcctcgcacttacagtcctcctggtcttcattcttaaatattcttgtcatagacgatcagcgcctaatggtgaagatggatgttacgtggatttgggaaaaggaaatttggaagttcaatttgaaggactttgtgctcgatatgcaaatgctgtaattaaacatccattgatatttgtttcacttggacttattgtagctgcagcttgttgctctggaaatttcaaatttcacagtctcacgcattcagttgatcaagtttcggctgccgatggagaaactagacggaacgagaaaaaattcatccattctttcggacctaatcatcgaattgagcaaatattcatcaacttaccacctaccactaagtctatgttcaacatgccactttttgaagaaatgttccaattggttgggaatattcagaatttgacagcttgctatgggaattcgtcagtgaagcttgatgatatatgctacaaacctatcggaaaaaatcacgggtgtgcaattatgagcccaaccaactatttccagaacaagtggaccaatttcgaaaacgcgggacctccaactattgatgatgagatctttgatgatcaacactgggaacatttgaagtactgtataagaaatcctcttaccgtatccacatactctgaaatgtcatgtttcggagagttttctgggcctatcgatccgattctagtgtttggtggaagtaatgaaagtatcaagggtgctgaaatgtactacaccgctcggactattatgataacggttttaatcagaggcccagaagatcaagctatagcctgggaaactgcgtttctaaacatgatgagtcgttatgaaatgaaacacgcaaattttacattcatgacagaaacgtctgttgcggaagaaattcatactgctgtggagactgataaaattgtgtctgtgattgcatgtgctgctgttcttatatgggttattacaatgcttggaattaatcattggccagagtcatccatattgtcagctctagttcatcacaagctactcatttcgatatcagctgttatgatcagtgtgatcagtgtttggtgctcgattggtatgttctcgctctttggtgtccatgcaacagataatgcaattgttgttttattctttgtcatcacttgtcttggaatcaatcgaatctttgtcattatcagaacatttcaagccaatggtcactgctacggacttccgaatatctcatatcgagaaatgaatcacagaatttcaaatgtaatgcgtcgaagtattccaattgttctaacaaactcccttatctgttcaacatgccttttcttagccggaggtgttcttccatatgtatctgtcagcatgccagcagttgaagtatttgcaagacatgctgggttggcaattcttatggatactgcattctatctccttgtaatgctaccactatttcaatatgatgcgagaagagaaatgtctggaaaatgtgaaatctggccgtggtacgagctatcaaacgagtcaaaaattaatctatgcatggaagcagttgatggaaatcttcgaagcccagtggattggttcaagcttgcgatcgcccctttattgctcaaaaagatttgtagaatttggatagctacattcttctttgtttccctaattatagcatgctactgtactctgtgcctcgaatttggtttcaatcaagttatggctttttcagagacaagttatctgacaaaacatttccaaaatatgaatgagaatttgaacattgggcctccgttatggtttgttgtagaaggtgatgtcaaatggcatgatccgaaaatgcagaacaagttttgtacattagccggatgtgatgataattcaatgggaaataagatccgatcattggcatacgctgagaactacaaaggaaactatctacacggcgatgtaaatatttggctggattcatatcttcagttcatgcatcctcgaggttcatgctgcaaaatggatggaaaacaattttgtgacccatcaaacgcaacacattgctcatcttgctcgtcgagcagcgtagcttcattgacaactacagaatatgagttctaccgtaatttgcatcacttcttggagactcctccatcaatccaatgtgctcatggtggaatggctctggcaaaaccagcgatcaatttgacaagaaatggaaaaattcaatctgcctatttttcaacatttttcaaaaagttgaatctctcggactctattcagctgtacgatgcctggagattcgcgaaatacctagcagacgacattgaacgtgaactggaaattcctggtgtgaaagtctacgtgtacagtaccttcttcccttattacgaacaatatctgactctgagcacaactgtatatactcttgtagttctggttctttttgttgctttcgtcacgatttccctatttttacgcgttaatttggctggaagtttagtcactgtatttgtactcctctcatcatatcttcacttgatggaatggatgtatctactcggaatcacagtgaatgtagtatcagttatcaatatggcaatgtctcttggaatcgctgttgagttttttgggcaaatgctacatggattctataactcaaaaaaaccgaaaagggaagaaagagcattcgctgcacttgtcagtaatggatctacgacattgtccggaatcttccctgctatcatgatcaccgctggatgtctttcatttgctgattctcgtgtgctcatcacatacttctgcaatcaacttgttggaattggtcttgtttgtgcagttcatggagttgtttacatgccaacacttttggcaattttcggatcggatttctatcagaatgtgagcagtgaagaagaatcaaccgatgaagcagaactgcaggatactcctccatcgaccacatcaagtacttccagcacttctgaaacatctgtctga

SEQ ID NO: 4 - protein, 1274 aa, Caenorhabditis elegans
MRQGGGGGERMVSVLFLLLIHLALCQAKCVMTECDGEEDSNHPPCKTNKSTYLPITVTRSLNPTYMARFEKYCSYLVQEEDKAQVCCTELQLKGMTDRISNAATILGSCPSCFDNFAKLWCQFTCSPDQSKFMKVMETTGPKNVVVKMEFKVNRDFVEGLYESCRHTWFANGLALRLMSLGGKVSFENFYGFMGTKNLAQSIPINTEFQFSRMKNAMNIPTTPCHKSAGPKVPACGAIDCPTNAHQLVDISKVEHLGTKVFHPHFPDFEWLLKICGCLALTVLLVFILKYSCHRRSAPNGEDGCYVDLGKGNLEVQFEGLCARYANAVIKHPLIFVSLGLIVAAACCSGNFKFHSLTHSVDQVSAADGETRRNEKKFIHSFGPNHRIEQIFINLPPTTKSMFNMPLFEEMFQLVGNIQNLTACYGNSSVKLDDICYKPIGKNHGCAIMSPTNYFQNKWTNFENAGPPTIDDEIFDDQHWEHLKYCIRNPLTVSTYSEMSCFGEFSGPIDPILVFGGSNESIKGAEMYYTARTIMITVLIRGPEDQAIAWETAFLNMMSRYEMKHANFTFMTETSVAEEIHTAVETDKIVSVIACAAVLIWVITMLGINHWPESSILSALVHHKLLISISAVMISVISVWCSIGMFSLFGVHATDNAIVVLFFVITCLGINRIFVIIRTFQANGHCYGLPNISYREMNHRISNVMRRSIPIVLTNSLICSTCLFLAGGVLPYVSVSMPAVEVFARHAGLAILMDTAFYLLVMLPLFQYDARREMSGKCEIWPWYELSNESKINLCMEAVDGNLRSPVDWFKLAIAPLLLKKICRIWIATFFFVSLIIACYCTLCLEFGFNQVMAFSETSYLTKHFQNMNENLNIGPPLWFVVEGDVKWHDPKMQNKFCTLAGCDDNSMGNKIRSLAYAENYKGNYLHGDVNIWLDSYLQFMHPRGSCCKMDGKQFCDPSNATHCSSCSSSSVASLTTTEYEFYRNLHHFLETPPSIQCAHGGMALAKPAINLTRNGKIQSAYFSTFFKKLNLSDSIQLYDAWRFAKYLADDIERELEIPGVKVYVYSTFFPYYEQYLTLSTTVYTLVVLVLFVAFVTISLFLRVNLAGSLVTVFVLLSSYLHLMEWMYLLGITVNVVSVINMAMSLGIAVEFFGQMLHGFYNSKKPKREERAFAALVSNGSTTLSGIFPAIMITAGCLSFADSRVLITYFCNQLVGIGLVCAVHGVVYMPTLLAIFGSDFYQNVSSEEESTDEAELQDTPPSTTSSTSSTSETSV

SEQ ID NO: 5 - DNA, 3999 bp, Homo sapiens
  CDS (1)..(3999)
atggcggaggccggcctgaggggctggctgctgtgggccctgctcctgcgcttggcccagagtgagccttacacaaccatccaccagcctggctactgcgccttctatgacgaatgtgggaagaacccagagctgtctggaagcctcatgacactctccaacgtgtcctgcctgtccaacacgccggcccgcaagatcacaggtgatcacctgatcctattacagaagatctgcccccgcctctacaccggccccaacacccaagcctgctgctccgccaagcagctggtatcactggaagcgagtctgtcgatcaccaaggccctcctcacccgctgcccagcctgctctgacaattttgtgaacctgcactgccacaacacgtgcagccccaatcagagcctcttcatcaatgtgacccgcgtggcccagctaggggctggacaactcccagctgtggtggcctatgaggccttctaccagcatagctttgccgagcagagctatgactcctgcagccgtgtgcgcgtccctgcagctgccacgctggctgtgggcaccatgtgtggcgtgtatggctctgccctttgcaatgcccagcgctggctcaacttccagggagacacaggcaatggtctggccccactggacatcaccttccacctcttggagcctggccaggccgtggggagtgggattcagcctctgaatgagggggttgcacgttgcaatgagtcccaaggtgacgacgtggcgacctgctcctgccaagactgtgctgcatcctgtcctgccatagcccgcccccaggccctcgactccaccttctacctgggccagatgccgggcagtctggtcctcatcatcatcctctgctctgtcttcgctgtggtcaccatcctgcttgtgggattccgtgtggcccccgccagggacaaaagcaagatggtggaccccaagaagggcaccagcctctctgacaagctcagcttctccacccacaccctccttggccagttcttccagggctggggcacgtgggtggcttcgtggcctctgaccatcttggtgctatctgtcatcccggtggtggccttggcagcgggcctggtctttacagaactcactacggaccccgtggagctgtggtcggcccccaacagccaagcccggagtgagaaagctttccatgaccagcatttcggccccttcttccgaaccaaccaggtgatcctgacggctcctaaccggtccagctacaggtatgactctctgctgctggggcccaagaacttcagcggaatcctggacctggacttgctgctggagctgctagagctgcaggagaggctgcggcacctccaggtatggtcgcccgaagcacagcgcaacatctccctgcaggacatctgctacgcccccctcaatccggacaataccagtctctacgactgctgcatcaacagcctcctgcagtatttccagaacaaccgcacgctcctgctgctcacagccaaccagacactgatggggcagacctcccaagtcgactggaaggaccattttctgtactgtgccaatgccccgctcaccttcaaggatggcacagccctggccctgagctgcatggctgactacggggcccctgtcttccccttccttgccattggggggtacaaaggaaaggactattctgaggcagaggccctgatcatgacgttctccctcaacaattaccctgccggggacccccgtctggcccaggccaagctgtgggaggaggccttcttagaggaaatgcgagccttccagcgtcggatggctggcatgttccaggtcacgttcacggctgagcgctctctggaagacgagatcaatcgcaccacagctgaagacctgcccatctttgccaccagctacattgtcatattcctgtacatctctctggccctgggcagctattccagctggagccgagtgatggtggactccaaggccacgctgggcctcggcggggtggccgtggtcctgggagcagtcatggctgccatgggcttcttctcctacttgggtatccgctcctccctggtcatcctgcaagtggttcctttcctggtgctgtccgtgggggctgataacatcttcatctttgttctcgagtaccagaggctgccccggaggcctggggagccacgagaggtccacattgggcgagccctaggcagggtggctcccagcatgctgttgtgcagcctctctgaggccatctgcttcttcctaggggccctgacccccatgccagctgtgcggacctttgccctgacctctggccttgcagtgatccttgacttcctcctgcagatgtcagcctttgtggccctgctctccctggacagcaagaggcaggaggcctcccggttggacgtctgctgctgtgtcaagccccaggagctgcccccgcctggccagggagaggggctcctgcttggcttcttccaaaaggcttatgcccccttcctgctgcactggatcactcgaggtgttgtgctgctgctgtttctcgccctgttcggagtgagcctctactccatgtgccacatcagcgtgggactggaccaggagctggccctgcccaaggactcgtacctgcttgactatttcctctttctgaaccgctacttcgaggtgggggccccggtgtactttgttaccaccttgggctacaacttctccagcgaggctgggatgaatgccatctgctccagtgcaggctgcaacaacttctccttcacccagaagatccagtatgccacagagttccctgagcagtcttacctggccatccctgcctcctcctgggtggatgacttcattgactggctgaccccgtcctcctgctgccgcctttatatatctggccccaataaggacaagttctgcccctcgaccgtcaactctctgaactgcctaaagaactgcatgagcatcacgatgggctctgtgaggccctcggtggagcagttccataagtatcttccctggttcctgaacgaccggcccaacatcaaatgtcccaaaggcggcctggcagcatacagcacctctgtgaacttgacttcagatggccaggttttagcctccaggttcatggcctatcacaagcccctgaaaaactcacaggattacacagaagctctgcgggcagctcgagagctggcagccaacatcactgctgacctgcggaaagtgcctggaacagacccggcttttgaggtcttcccctacacgatcaccaatgtgttttatgagcagtacctgaccatcctccctgaggggctcttcatgctcagcctctgccttgtgcccaccttcgctgtctcctgcctcctgctgggcctggacctgcgctccggcctcctcaacctgctctccattgtcatgatcctcgtggacactgtcggcttcatggccctgtgggacatcagttacaatgctgtgtccctcatcaacctggtctcggcggtgggcatgtctgtggagtttgtgtcccacattacccgctcctttgccatcagcaccaagcccacctggctggagagggccaaagaggccaccatctctatgggaagtgcggtgtttgcaggtgtggccatgaccaacctgcctggcatccttgtcctgggcctcgccaaggcccagctcattcagatcttcttcttccgcctcaacctcctgatcactctgctgggcctgctgcatggcttggtcttcctgcccgtcatcctcagctacgtggggcctgacgttaacccggctctggcactggagcagaagcgggctgaggaggcggtggcagcagtcatggtggcctcttgcccaaatcacccctcccgagtctccacagctgacaacatctatgtcaaccacagctttgaaggttctatcaaaggtgctggtgccatcagcaacttcttgcccaacaatgggcggcagttctga

SEQ ID NO: 6 - protein, 1332 aa, Homo sapiens
MAEAGLRGWLLWALLLRLAQSEPYTTIHQPGYCAFYDECGKNPELSGSLMTLSNVSCLSNTPARKITGDHLILLQKICPRLYTGPNTQACCSAKQLVSLEASLSITKALLTRCPACSDNFVNLHCHNTCSPNQSLFINVTRVAQLGAGQLPAVVAYEAFYQHSFAEQSYDSCSRVRVPAAATLAVGTMCGVYGSALCNAQRWLNFQGDTGNGLAPLDITFHLLEPGQAVGSGIQPLNEGVARCNESQGDDVATCSCQDCAASCPAIARPQALDSTFYLGQMPGSLVLIIILCSVFAVVTILLVGFRVAPARDKSKMVDPKKGTSLSDKLSFSTHTLLGQFFQGWGTWVASWPLTILVLSVIPVVALAAGLVFTELTTDPVELWSAPNSQARSEKAFHDQHFGPFFRTNQVILTAPNRSSYRYDSLLLGPKNFSGILDLDLLLELLELQERLRHLQVWSPEAQRNISLQDICYAPLNPDNTSLYDCCINSLLQYFQNNRTLLLLTANQTLMGQTSQVDWKDHFLYCANAPLTFKDGTALALSCMADYGAPVFPFLAIGGYKGKDYSEAEALIMTFSLNNYPAGDPRLAQAKLWEEAFLEEMRAFQRRMAGMFQVTFTAERSLEDEINRTTAEDLPIFATSYIVIFLYISLALGSYSSWSRVMVDSKATLGLGGVAVVLGAVMAAMGFFSYLGIRSSLVILQVVPFLVLSVGADNIFIFVLEYQRLPRRPGEPREVHIGRALGRVAPSMLLCSLSEAICFFLGALTPMPAVRTFALTSGLAVILDFLLQMSAFVALLSLDSKRQEASRLDVCCCVKPQELPPPGQGEGLLLGFFQKAYAPFLLHWITRGVVLLLFLALFGVSLYSMCHISVGLDQELALPKDSYLLDYFLFLNRYFEVGAPVYFVTTLGYNFSSEAGMNAICSSAGCNNFSFTQKIQYATEFPEQSYLAIPASSWVDDFIDWLTPSSCCRLYISGPNKDKFCPSTVNSLNCLKNCMSITMGSVRPSVEQFHKYLPWFLNDRPNIKCPKGGLAAYSTSVNLTSDGQVLASRFMAYHKPLKNSQDYTEALRAARELAANITADLRKVPGTDPAFEVFPYTITNVFYEQYLTILPEGLFMLSLCLVPTFAVSCLLLGLDLRSGLLNLLSIVMILVDTVGFMALWDISYNAVSLINLVSAVGMSVEFVSHITRSFAISTKPTWLERAKEATISMGSAVFAGVAMTNLPGILVLGLAKAQLIQIFFFRLNLLITLLGLLHGLVFLPVILSYVGPDVNPALALEQKRAEEAVAAVMVASCPNHPSRVSTADNIYVNHSFEGSIKGAGAISNFLPNNGRQF

SEQ ID NO: 7 - DNA, 972 bp, Caenorhabditis elegans
cctgtgttgcggattcgccaagtgcgcagcgaccaccccagcggcgttgtcgatttcaacaactagcgtgatattggttttcgtggcataaagtacttgatcaaaaatagtcgactttctgaacgggaagtaaaaaaaaaatacaaacaaaacagatttcttataagttacaaataaataatggtgaattcaagttaatttagaaacaattaacacacaatcacagtaccggacgtttgtgagaccttatagtccagatttgggaattactgacaaccgaataatacgcagaacactggaaaatgaaaaagttttagggatgccgcggatttccaactaatcagatttttgctagacgtacaattaatgttaagttagaataatgtcaaaacatctggtgaagtttgtgaataattgggtttttaatgtttagttgttttaagttttaacatttgttagttctatcatatttttttggattcagttttagtttttgtgatcctgtgtgcccaattgaggaaaactggttgcgttttaggtgcctacaatacaattttaccaagttttttttgcagatttttctatcctgtcggtcacaatgcaactagcaagtaaaaaagtaatgactttgttttaaataccatgtgctagctgagtttcatcatgtccagaatcacctcatcgttaaaaagtcatgagtatcaaagtgcagtgtgatcaaactgtattacttgaaacaatttagttatatgtttagaacccctcattcaaaattaatagacagggctctcaccgaatgttgcaatttgtttctgataagggtcacaaagcggagcgaatgcttgaatgtgtccatcaatgagcttatcaatgcgctaaaacgctataacttccatatgaagtcaatcgaacatatgtcaatctttagccgtatataaaggtgcactgaaaacagtccaatcacggttcagc

SEQ ID NO: 8 - DNA, 1643 bp, Caenorhabditis elegans
ttagtaatctcatacatataaagatgacattttcctacatatgtataaaaattgcaaaaagtgaaacttcaaatatccatattctaagactttttttcaatctgctccaatttctcaaatctgtttaaatttttgacaaatgttaatcagttttcggctatttttgatgtcacttggtcttagagcttatatagcttaacagtcaaaaaagttacgcacttggaacaaaaaatgctctaaaatttccagataagaattggaaaggttttgatttgttcctttcatgaattgtggttttcaattgtttgttttaaaactattttcaaaaatgcagcttgcgatcattcatgtgttgttcgtagtttgtgacgtcaattttaaaatcttcaagaaatacttgcatttctataaaattgatttgaaattttgaagcctaaattactgtcaaaaattttgattcgaaggttttctaaaacaatgagtgatttcgggcgttatatcaaatatccaaatatataaaatttcataaaactactataaactatatgctagaaactcgtatttatactagaaatactagaaactagaaactataaaaacctggaacatcaaataaatttaatgtcagactacaaccctttaaactacataccaacgatttattagtttgagcttcaagaattttatattcttaagagggacgcacaaaaaatcactcttcacactatttctttgttgaatagaggtcagcggcttttaaaaattataatttgaaaaaaaattgggactttttcaaaaaaaaagcttccaaacgtccctattaggaaatgtaaacttattcccaaaaaattaaaaattccagagaaagtagacaaatttcagaaaacttaccgcgctacatctaatactttctcataggtttttttattgggaaactgatgaaaattatttgaattcataataaaataaattcgtatttagcatttgaaaatttgcaccaatgtattatttaattttttttttcgaaaatttaacgcattttctctctaaaaactcgaaatttagtgtgttctaaacaacagtaagcatacaatacctttgttcaaaattgacgtgctttctgaaccaatatggttagtttcaaaaatttttgtattataggatagaaatatttggaaataaattttaaaaccaaacttatgcctttctcttttagtatcccagctaggtaatattttagtatttgcccaaatccttgaagtaaggagtatataatttttgaaaaacaataaaactccagataattcatagttttttctcgaaagaaaatttttgagattagttattgaacttcatttttgaacattattcgttgaaaaacactcgctttgtctttattttcaaaaaaattccgatttccccaaccagaaaaaaaaacagatagaagaaatttctccttaaatttcattgtccatctctcttggaaacacattatctatcaaatgaaaaacgcattttttttcctggcagaaaaatgaaattggttagattacactggttaggtttgaaggtgtaactttcgctttctcagcaactttcagtataaaaggaaacggtcaccatttagaaagacatcagttcat

SEQ ID NO: 9 - DNA, 4185 bp, Caenorhabditis elegans
gatcttctccttcccatgtgctgcaaaccacaacttccggcagccacggaggtatgtgataagttgaatgagtcatcatgacgtttgaaaaattacaacgaattgattaatcgacaaatttcagcagattcaaccatttcgtcaacaacatctttggcatcacctgtcactttaccaatgactccatcttcagattcttctactccaatgcacgccatcgaacccgttcagcatctctaatttcgaaatgtatgaactccaattcttgataaccaattcctgactctgtaactattattattataatcctattacccaccttttttcccattctgctcttagttctcccccaacccgtacattttctcaactaataaagacaatgagaaagtgagagagaataagacgaactgaaaagaatgtgtaaacagtttattgcctctcgattgtaccatctagtgtcttgtcgtgtgcataactactgataacagatcgaatgtgaaagacccaataaatgcttggaaagtatcaaatatgtttagaaatagtatttagagacttatgtgggacgaaaaaacaggagcattaagaatacagttgcaaacattcatcaagttataatctaataatataaattcaaaaatgttaatgataaaaaatgatgatcaaagtaggaaattagtggcgagaaaacaataaatcatcagaaaatgactgatcttgagtagagtgctttttttgtagatttctgattctaaaactgaaaaaaaaactaaaaatgctcgatgagtcatactaattgaaaaataatcagattgaaaacgttaaaaatgtacttgctaacatacttccatggttatttttgaacacaaataaagttatatacatctttaccggcaccagaagaaatcaccgctaaccaatgttcagtttttacctgaatatccctgaaagtacaaaaaaatcaatgttcaatcaagtttttgcatgccttcgtgtgatggctcgcaccaagtattctgacagttataaaatgtttcctgtgatcatgaatttgaaaaaaaaagaactgatgggaaaatgtgatattcataaatgaaatgtttaataaacaaaaaagaaccgtcatcacaaatctactctgaaacgttcacagttcctaattgaccaattcagaattttttgtactgacttaataaatataaatttacagttttaagaaacataaattgaattatataaataaaaaaaaaaaaactaaaatgaagacgtgatggaatgtttcacgtattaaaaaagctctcggaccgtttgacatccagaaattatgtgaaagtgattttctaaccctttgacgtcaaaaaagtttttcttttttgaaagatacgctgtttagaaatacttatacagctgattttagaatctccaaaaaaccttcaattacttaacaaactttctaaataaagaattttgattttgattttctttgaacttcaaaccccccgttaaaaaaccaaattaaaaaaaagataccagattaattttaaagtgaaaaatataaatttattcccgactgttaaaaacggtttataaacggagtcacaacactcatatacaactatcgacaccaaataaataacttcgaaagctaattgtgtcatttcggatatagagagtagtgtgagcagatgtgtttttgcagaactaaattgatgacactattatggaatataatgaccaaatgttaaatgtgttaaggtttgatatcaaaacctgtattttctttttatacagaattgataatgttatcttcaattgatttctacttctgagctacggcgatacgaggacgcattctcaacgataatgttgccattttgtcctgataatttttttactgattgtttcagaacacccatagtttttctctattaaacgttcatccttgacttccccgagtttgctggctgaataggaaatttgaagacaaaaaggaaagaatcggctcaaacgtcatgcaactgataaggcgaccgtacttcatttcaaaagaagctcacttactgagcgcaaacattgagaaatgagagcaaaagaagtggttttacaacatgctaatgtttttagaccttgtacccaataatattactgtagtatacagttcggagagcatatggttgaaatcttgaaataccaatttatcactagtttgattgtgttatcgatgtataaagatatattttatcatttttgattattatctgatattgtggggtgtgaagtaatattatgtgcgtgtgtggctgattatcgaaaaaaactgaaaattatcaatttttctacaggttatctttttttgttttatttttcattattgtattcttcatactccttatcctgcccgaatcacaaaagttccagacaacttgaaggtcgcatcacgttttgtttataagaatgttgaactggtcagctttggagacagaactgtcctccaaatgttgaatatgtttccgtaaaacacactgtgtaacaaaaagcgtaccctttgatccggtattcttgaaaaaaaacatagaagggcgtacttttcatatctcacaaatatgcgttttttattttataataatatcatttattgataattatatgtatttgtatgttttcttgtctttaaaattatggtgtgacatcacagtttaaaaattatatgaaaactaatgagtgaaaagttaaaaatgtatagtttgccgtattttctccattagtattgtattcaaaacttatttccaattggtgagcactataaactttgtagggtaattgaggttcaatactaatttttgaatatctagcttttgacaaattattcaagttttgacaatgatgccttatcaaattaaaaaaaattaccataaattcttgaaagcttttgattttgtttcactctgtgaaggatagtaatttatcaacattttaactagatacatctgttctggcaataaattaaagcaagactattactatgtgcagtattaatagctgcattgcaacttgtttgaaagtttcagtgaaaacgcaaaaactcaatttaattttcaagtttgtttgcaaaattggatatatgtatccatcaaaagtctgatcgcctgttttatctacttattgtctcctcagttgatttttttgtagttattataaacgctactttgaaaccaatattttcttcctcattccacttttttcaattaccaactaccgtacatgcaatgatgggcggacccgagtcccatgtttggctggaagtgggtggttgtctgcgtatatgagcgacagaggtcggggctgaaactgataagaatagtcgacactaacgccataatcgctagccagccatcatgcacaccgagctcggtgtgcacaccatctttcttttcaaaccaatacgctttgtgccttcattgacaattttctttttgataaaatcagcctatctatacttcccaatcatttttagtcttatcgttgaacagctatcgaggtgccactgttttcactgatatcttctaagttactatggcattaacatctttgtgttcttattttatgggttattttaattaatttttgcagttaatttttggaatgagcaagaaaatgttaattgtaatatcttcgtctgaaaattgtcttcaaatagttttaattttaaaggcagtatttaagaaaatacacttctcgaagcatttttgaatttttgaatttttaaactgcttgatgttttaggtgccactgttttcattgataagttttgatgtataaatgcttgattttcttggcattctaataaaatagaaactagaaaatagattatagaatggataataactacaatgataatgtcaacggctgggccgaaatggaaccatctcaaccaatgggaggtctgcgcctaccaactcagaacatggatccaccagagcaaaataatgagtcacaattgagtgaactaccgagaagtaaacaaaacactgaaatgcttgtggattataaaattataaaatttcagtgaaaattgataatgattacgcatctccaattgaacggcaaagtgttatcacaagtggcacaaataactatgagccgaaagtggaaactgttacatcatgtaagtgtgaattcaag

SEQ ID NO: 10 - DNA, 1852 bp, Caenorhabditis elegans
aagcttaatgaagtttatttcagatcagtaattcgaaatgtttctactggaatccgccaaattgtcgacaactcttttaagaaaacgactaaaatgaacttgagtgttagcggcgtcttcaccaatacctttagtgacgatgtcgaagttgactataactgggacaatatcatcgaatattcttacaaatcttcggaaacggagtataagaaaagagaggacgatggagaccatacggaaatagctgttagagagctcggaattcagctcaacaatccgaatcatcagctggagtacctggaattctggataaaacctttacgatattttaccgaagaagaaaggaccgaacgatggaattgtctggaaaaattcttcaagaaggttgagaactcgaaggttgctaaaatctcgttcgttggattcaggggatataaaattgcaacgattttgcaatctttctcaccaggaaccctgacacgactttatgtcaatgttgctgattcggatatctctcaaatcatcgacgcggagcattggatttatctaaaaaagatcagagtgctttcgccttctccggtttccattccgattgatgctattcttcacatgacacacttcgacgtttctgtgatccacctttccactgatgatgtcgcaaagatcaaaaatgtgagtgaacaataaaaagttgaaaaaacattattttaaaataattactggtcgttctcgaatccgcaagtttcacaatggggatgatccgtaccaacatcacggacccaatggaaactgcaaaaatcttcggaacgcacattcatcgtgatgatgtggatgttttgggtgacatggatattaacaaggacgatggtccagaagttcatttggatgacgattggcatgaggaggagctcgtgggggctgtgggggcggaggaggaggaggacgaggaggacgaggaagaggaggacgaggacgaggaagaggggatggctgatgtcccggaagtcaatgaggccgtgcacagagtcaacattaaactcggcgacgctgacatcaccgttgtggtgtctccgacatacttgaagattgcaaagaatactcgtgaaaattgaactttaattcctttctctttttttttgactcaaaaaataaatagtatttcaaaggaatcgcatttcaaactgatacttaagtttttcaataggttttttttttcagttttgatttttttttgtgcttaaagagaagactgttagccactcagccacttcagccatatcgaaacagtcatattggctctctttcagatcacttaaaactccgaactatgatgacgaaaaaatgttttcttgaattttcattttattgtgaaattgaaaactctgcatagcccgcagtcacttgaaagcacatttaagatggaatcaaatggtaagaaagctcgtgtaatcaggagattttatccggggtatgccgaggaatatatggtgcactcgcattagctggcattttttgtaattatcggatttctacctttttcttaatctagtttgttatcattgtccatcatttacaaccaaaaatttgcgtcatttacaaccaaaaattgatctgacaattatcataattcggtttttattttcagaagaaacgctccgatgtgatcctatagtgaatatacttttccatcctctctgaaataaacaattatgaagaatgcatcaatttgcattcgaaataactgccgtattttgtctattggtatggctgcagtaccatttttcttcgcgtctttgcgcagccttcgggcgctaccaataaggctaagccggggtaagttatttcagaattc

SEQ ID NO: 11 - DNA, 35 bp, Artificial Sequence
  ncr-14kbsph5′ primer
gggggcatgccacaacaattatctttatcctaact

SEQ ID NO: 12 - DNA, 36 bp, Artificial Sequence
  ncr1pBam3′ primer
ggggggatccttcttgtgcatcgactgaaacatacg

SEQ ID NO: 13 - DNA, 27 bp, Artificial Sequence
  ncrp2245 primer
ctatacatttatgcctcagagcaatca

SEQ ID NO: 14 - DNA, 32 bp, Artificial Sequence
  ncr-23′prom primer
tccggaaatgtagaaatttaatattaaatact

SEQ ID NO: 15 - DNA, 38 bp, Artificial Sequence
  GFP5′Xba primer
ggggtctagaatgagtaaaggagaagaacttttcactg

SEQ ID NO: 16 - DNA, 42 bp, Artificial Sequence
  GFP3′Not primer
ccccgcggccgcctatttgtatagttcatccatgccatgtgt

SEQ ID NO: 17 - DNA, 27 bp, Artificial Sequence
  ncr5′sma primer
cccgggaaacaactactcattttttgc

SEQ ID NO: 18 - DNA, 24 bp, Artificial Sequence
  ncr13′A primer
gatttatgtgttctacttatgttc

SEQ ID NO: 19 - DNA, 24 bp, Artificial Sequence
  ncr-2noATG primer
cgtcaaggaggaggaggaggcgag

SEQ ID NO: 20 - DNA, 28 bp, Artificial Sequence
  ncr-23′UTR primer
ctgaaatcggataaataaattaataaat

SEQ ID NO: 21 - DNA, 24 bp, Artificial Sequence
  ncr-13′intron1 primer
cgatactaatgtggagcccacagc

SEQ ID NO: 22 - DNA, 25 bp, Artificial Sequence
  ncr-15′ intron1 primer
cgaagcacgacggacatcgtcccag

SEQ ID NO: 23 - DNA, 23 bp, Artificial Sequence
  F09G8F2438 primer
gattggttcaagcttgcgatcgc

SEQ ID NO: 24 - DNA, 28 bp, Artificial Sequence
  F09G8R5969 primer
gagcacattggattgatggaggagtctc